(19) United States
(12) Patent Application Publication
(10) Pub. No.: US 2019/0105460 A1
(43) Pub. Date: Apr. 11, 2019

(54) COMPOSITIONS, SYSTEMS, METHODS, AND ARTICLES FOR RETRO-NASAL DELIVERY

(71) Applicant: Sensory Cloud, LLC, Cambridge, MA (US)

(72) Inventors: David A. Edwards, Cambridge, MA (US); Rachel Diane Field, Huntington Beach, CA (US)

(21) Appl. No.: 16/122,673

(22) Filed: Sep. 5, 2018

Related U.S. Application Data
(60) Provisional application No. 62/556,974, filed on Sep. 11, 2017.
(60) Provisional application No. 62/628,395, filed on Feb. 9, 2018.
(60) Provisional application No. 62/652,069, filed on Apr. 3, 2018.
(60) Provisional application No. 62/687,970, filed on Jun. 21, 2018.

(51) Int. Cl. A61M 21/00; A61M 15/00; A61M 15/08; A61K 9/00; A61K 9/107; A61K 9/127

(52) U.S. Cl. CPC A61M 21/00; A61M 15/009; A61M 15/08; A61K 9/007; A61M 2210/0618; A61K 9/127; A61M 2021/0016; A61M 2202/0482; A61M 2205/123; A61K 9/1075

(57) ABSTRACT

A system to provide active agent (e.g., scent molecules) includes a delivery system that may include a reservoir, an actuator (e.g., nebulizer), and a fluidly communicative scent path through which a vapor or aerosol may be supplied or dispensed, the vapor or aerosol comprising readily-soluble droplets having a median size range of approximately 2 microns to approximately 50, 20, or 10 microns for introduction to an animal via a retro-nasal route. The vapor or aerosol may be dispensed below a top of the vessel, in a direction that at least initially retains the vapor or aerosol in the vessel. The system may include a base to support or secure the vessel, a cover to close the vessel, and, or a conduit or straw to sip from the vessel. The system can include switches or sensors to provide signals to control operation of the actuator, for instance, based on position, orientation, or direct or indirect user input. The system takes the form of conduit, e.g., straw or stirrer.

### BACKGROUND

### Field

This disclosure generally relates to providing therapeutic and other substances in a form suitable for retro-nasal delivery, which may be used to modulate human or other animal biological processes or treat medical conditions, and in particular to related compositions, systems, methods, and articles of manufacture.

### Description of the Related Art

All of our five sense act as messengers that deliver information to the brain, which then processes this information, causing us to respond in relatively predictable ways. Within the context of our sense of smell, all odors present themselves in specific chemical configurations, allowing humans to perceive a wide variety of distinct odors. Odor perception initiates in the nose, where the respective molecules are detected by a large family of olfactory receptors. Olfactory receptors have diverse protein sequences, and are assigned to subfamilies on the basis of sequence relationships. These observations formed the basis for research into the mechanisms underlying human odor perception, leading to the 2004 grant of the Nobel Prize in Physiology and Medicine to Linda B. Buck and Richard Axel.

However, even given the significant importance of our sense of smell, relatively little has been done to develop the apparent physiological value of this sense or to more thoroughly incorporate it into how humans experience the world around them on a daily basis. Although some systems and devices have been proposed for attempting to provide olfactory sensations to users (see, for instance, U.S. Pat. Nos 8,050,545, 8,032,014, 6,654,664 and 6,803,987), they have proven inadequate as mobile, personal, targeted and effective delivery systems that may be used to alter behavior.

As explained herein, new approaches that effectively deliver therapeutic and other substances in order to elicit a physiological response are desirable.

### BRIEF SUMMARY

Recent advances in olfaction biology have made it clear that flavor images that appear in the brain as a consequence of activating sensory receptors in the process of eating and drinking play a role in up- and down-regulating of metabolic function. Among the most important of sensory receptors involved in the creation of these flavor images are olfactory receptors in the nasal epithelium. Such receptors appear elsewhere in the body, including the heart, gut, and circulating cells of the immune system. As such, stimulation of olfactory receptors can influence not only metabolic processes but other processes including those involved in immunity and brain function.

The delivery of substances (i.e., active substances) to the nasal epithelium to modulate human health, as in the delivery of active substances for relieving congestion, or symptoms related to asthma, generally involves the delivery of dry or liquid formulations to the nose via a nebulizer, metered dose inhaler, or dry powder inhaler. These delivery modalities conventionally involve spraying or sniffing active substances directly into the nose via the nostrils or nasal vestibule.

Active substances deposit in the nose, depending on the nature of the delivery system and technique, with some associated degree of efficiency. This efficiency can be measured as a fraction of “delivered dose” to “nominal dose.” Delivered dose is the mass of active substance that not only deposits on the nasal epithelium, but is delivered to the target tissues and/or receptors. Given that clearance of the active substance from the nose is rapid, delivery to the nose of the dose of active substance in a form that is quickly dissolved and distributed is highly desirable.

Naturally, delivery of odorants (i.e., scent molecules) to the nasal epithelium occurs in two ways. The first, ortho-nasal scent delivery, occurs by sniffing odorants in the atmosphere, e.g., directly via the nostrils or nasal vestibule. The second, retro-nasal scent delivery, occurs by the natural diffusion and convection of odorants in the mouth into the nasal passages via the oropharynx. This latter delivery is referred to as retro-nasal olfaction, and is promoted by exhalation.

It has recently been found that many people who cannot perceive scent via ortho-nasal olfaction, can actually perceive scent or flavor via retro-nasal olfaction. The surprising “special capacity” of retro-nasal olfaction relates to the fact that the human oropharynx is supremely well designed to bring odorants in the mouth into the nasal passages. As a consequence, flavor perception plays a critical role in the regulation of human metabolism. Humans develop likes and cravings for certain foods as a consequence of experiencing the metabolic effects of these foods, and associating these effects with flavor images in their brains. Eventually, these images, as memories (the olfactory nerve links olfactory receptors in the nose with the seat of long-term memory, the hippocampus) drive food interests and cravings that lead to humans receiving the metabolic effects they enjoy.

Recently, human and animal studies have found that simply perceiving the scent of certain foods, like chocolate or the aroma of roasted coffee beans, can trigger metabolic effects that heretofore have been believed to occur only on the ingestion of chocolate or coffee. This surprising finding, combined with the discovery of the general efficacy of retro-nasal olfaction versus ortho-nasal olfaction, opens up a completely new opportunity for active substance (e.g., drugs, and various scent molecules that have until now principally been understood to relate to food and flavor perception) delivery to the nose.

Described herein are new compositions, apparatus, methods and articles for delivery of active substances to the nose. The described compositions, apparatus, methods and articles can be employed for the up- and down-regulation of human (and other animal) metabolism, as well as to other beneficial physiological effects, for instance decongestion. Rather than deliver active substances to the nose via the standard ortho-nasal route, the described approaches advantageously deliver active substances to the nose via the retro-nasal route. The active substances are formulated in readily-soluble water droplets that have a median size range of 2-50 microns, 2-20 microns, or 2-10 microns, advantageously too large for significant penetration into the lungs, while small enough to be carried into the nose.

As also described herein, apparatus are provided which allow the portable, discrete delivery of active substances, enhancing or efficiency of delivery to humans and other animals. Advantageously, the apparatus is configured to be portable, allowing the user to have the benefit of retro-nasal delivery, on demand, in a wide variety of environments. The apparatus and compositions may be used to enhance the efficiency of delivery of active substances, and to provoke a physiological response in a human or other animal via the connection of the olfactory sensory system.

In some implementations, a device is provided which takes the form of a vessel or at least includes a vessel portion, that in use holds a liquid. The device also includes a scent media reservoir that is distinct from an interior or chamber of the vessel, and which in use holds scent media. The scent media is used to emit scent into the vessel, for example as a spray of droplets. Use of a scent media reservoir that is separate from the vessel advantageously allows creation or generation of clouds of flavor/scent above food and drink, the clouds which differ from the aroma of the drink or food itself, and can be useful for conditioning appetite.

The scent media reservoir or outlet (e.g., port, nozzle) thereof may be positioned and oriented so that a spray or other distribution of scent media is directed towards, and optionally against, the inner surface (e.g., side wall) of the vessel, to advantageously ensure that the dispensed scent at least initially stays in the interior of the vessel, for instance until sampled or “ingested” by a human end user. The vessel may have an opening, In some implementations, the device may include a cover, the cover removably securable to the vessel, for example to selectively alternatingly provide and deny access to an interior of the vessel from an exterior thereof via the opening. In some implementations, the device may include an elongated hollow conduit (e.g., a straw), that provides a fluidly communicative path from an interior to the vessel to an exterior thereof, and which allows for a user or consumer to “sip out” or imbibe the “cloud” which is otherwise contained in the interior of the vessel. The device may include a nebulizer, for example a nebulizer comprising a screen and a piezo-electric element, solenoid, or an electric motor physically (e.g., mechanically, magnetically) coupled to move (e.g., oscillate, rotate) the screen and thereby cause dispersion of the scent media in the interior of the vessel, for instance as a spray.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a side elevational view of a delivery system that includes a reservoir that in use contains an active substance or composition, preferably formulated in readily-soluble water droplets that have a median size range of 2-50 microns, 2-20 microns, or 2-10 microns, and is operable to deliver the active substance or composition to an interior portion of a vessel, according to one illustrated implementation.

FIG. 1B is a side elevational view of a delivery system that includes a reservoir that in use contains a capsule and is operable to deliver an active substance or composition, preferably formulated in readily-soluble water droplets that have a median size range of 2-50, 2-20, or 2-10 microns, to an interior portion of a vessel, according to one illustrated implementation.

FIG. 2 is a dotted line representation of a front elevational view of a delivery system that is physically coupled to a wall of a vessel, according to one illustrated implementation.

FIG. 3 is a side elevational view of a delivery system in which a port is positioned to direct an aerosol or nebulized cloud of active substance or composition exiting the scent port at an angle above the horizontal, according to one illustrated implementation.

FIG. 4A is a dotted line representation of a front elevational view of a delivery system physically coupled to a side wall of a vessel in which the delivery system includes a selectively removable, rotatable cover for the port in which the cover is shown in an open position, according to one illustrated implementation.

FIG. 4B is a dotted line representation of a front elevational view of a delivery system physically coupled to a side wall of a vessel in which the delivery system includes a selectively removable, rotatable cover for the port in which the cover is shown in a closed position, according to one illustrated implementation.

FIG. 5 is a side elevation view of a delivery system that includes a selectively removable, twistable cover for the port in which the cover is shown in a closed position, according to one illustrated implementation.

FIG. 6 is a side elevation view of the delivery system of FIG. 5 in which the selectively removable, twistable cover for the port is shown in an open position, according to one illustrated implementation.

FIG. 7A is an isometric view of a bottom portion of a container in which a delivery system is incorporated onto a side wall of the container, according to at least one illustrated implementation.

FIG. 7B is an isometric view of a container with a lid that has one or more apertures in which a delivery system is incorporated onto a side wall of the container, according to at least one illustrated implementation.

FIG. 8 is an isometric view of a plate with an open top in which a delivery system is incorporated onto a side wall of the plate, according to at least one illustrated implementation.

FIG. 9 is an isometric view of a device including a vessel with an open top, and a base that includes electronics and on which the vessel is removably or detachably supported, according to at least one illustrated implementation.

FIG. 10 is an isometric view of a portion of the device of FIG. 9, including a nebulizer which can include a screen and at least one of a piezo-electric element, solenoid or electric motor physically coupled to move the screen, the device also including one or more of a radio, a transducer or sensor and a switch communicatively coupled to a control system, for example a microcontroller and memory, and operably coupled to control operation of the nebulizer, according to at least one illustrated implementation.

FIG. 11 is an isometric view of a portion of a device including a vessel with an open top, at least one scent media reservoir, and at least one nebulizer, the nebulizer or a port thereof positioned and oriented to dispense or disperse scent media in a direction that at least initially substantial retains the dispensed or dispersed scent media within an interior of the vessel, according to at least one illustrated implementation.

FIG. 12 is a top plan view of a portion of a device including a vessel with an open top, and three one scent media reservoirs and associated nebulizers distributed about a periphery of the vessel, the nebulizers or a respective port thereof positioned and oriented to dispense or disperse scent media in a direction that at least initially substantial retains the dispensed or dispersed scent media within an interior of the vessel, according to at least one illustrated implementation.

FIG. 13A is a side elevational view of a device including a vessel with a cover positioned to close a top or opening of the vessel, a conduit or straw that provides a fluidly communicative path through the top, and a scent media reservoir and associated nebulizer positioned and oriented to dispense or disperse scent media in a direction that at least initially substantial retains the dispensed or dispersed scent media within an interior of the vessel, according to at least one illustrated implementation.

FIG. 13B is a side elevational view of a device including a vessel with the cover removed to open the top or opening of the vessel, providing access to an interior thereof, a conduit or straw physically coupled to, or forming part of, the cover that provides a fluidly communicative path through the top, and a scent media reservoir and associated nebulizer positioned and oriented to dispense or disperse scent media in a direction that at least initially substantial retains the dispensed or dispersed scent media within an interior of the vessel.

FIG. 14 is an isometric view of a pair of cartridges that carry scent media and which are sized and dimensioned to be removably receivable by a scent media reservoir to supply scent media via the nebulizer, according to at least one illustrated implementation.

FIG. 15 is an isometric view of a delivery device to deliver a vapor, a cloud, or an aerosol comprising active scent media over the contents of vessel, according to at least one illustrated implementation illustrated along with a scent media cartridge, in which a scent media receptacle is located at a proximal portion of the delivery device and a switch is located at a distal portion of the delivery device.

FIG. 16 is an isometric view of a delivery device to deliver a vapor, a cloud, or an aerosol comprising active scent media over the contents of vessel, according to at least one illustrated implementation illustrated along with a scent media cartridge, in which a scent media receptacle is located at a proximal portion of the delivery device and a switch is located at the proximal portion of the delivery device.

FIG. 17 is an isometric view of a delivery device to deliver a vapor, a cloud, or an aerosol comprising active scent media over the contents of vessel, according to at least one illustrated implementation illustrated along with a scent media cartridge, in which a scent media receptacle is located at a proximal portion of the delivery device and a switch is located at a distal portion of the delivery device.

FIG. 18 is a schematic drawing of a delivery device to deliver a vapor, a cloud, or an aerosol comprising scent media, the delivery device including plural vials, each vial holding a respective distinct scent media in liquid form, according to at least one illustrated embodiment.

FIG. 19 is a schematic drawing of another delivery device to deliver a vapor, a cloud, or an aerosol comprising scent media, the delivery device including plural vials, each vial holding a respective distinct scent media in liquid form, according to at least one illustrated embodiment.

FIG. 20 is a schematic drawing of a delivery device to deliver a vapor, a cloud, or an aerosol comprising scent media, the delivery device including a spacer for holding the cloud, according to at least one illustrated embodiment.

FIG. 21A is an exploded view of a delivery device to deliver a vapor, a cloud, or an aerosol comprising scent media, according to at least one illustrated embodiment.

FIG. 21B is a perspective view of the delivery device to deliver a vapor, a cloud, or an aerosol comprising scent media of FIG. 21A, according to at least one illustrated embodiment.

FIG. 21C is a side view of the delivery device to deliver a vapor, a cloud, or an aerosol comprising scent media of FIGS. 21A and 21B, according to at least one illustrated embodiment.

FIG. 21D is a cross-sectional side view of the delivery device to deliver a vapor, a cloud, or an aerosol comprising scent media of FIGS. 21A-21C, according to at least one illustrated embodiment.

FIG. 21E is a side view of components of the delivery device to deliver a vapor, a cloud, or an aerosol comprising scent media of FIGS. 21A-21D, according to at least one illustrated embodiment.

FIG. 21F is another side view of components of the delivery device to deliver a vapor, a cloud, or an aerosol comprising scent media of FIGS. 21A-21D, according to at least one illustrated embodiment.

FIG. 22A is a rear view of a printed circuit board and associated components coupled thereto for use in the delivery device to deliver a vapor, a cloud, or an aerosol comprising scent media of FIGS. 21A-21F, according to at least one illustrated embodiment.

FIG. 22B is a side view of the printed circuit board and associated components of FIG. 22A, according to at least one illustrated embodiment.

FIG. 22C is a front view of the printed circuit board and associated components of FIGS. 22A and 22B, according to at least one illustrated embodiment.

FIG. 22D is a perspective view of the printed circuit board and associated components of FIGS. 22A-22C, according to at least one illustrated embodiment.

FIG. 22E is a rear view of the printed circuit board of FIGS. 22A-22D, without the associated components coupled thereto of FIGS. 22A-22D, according to at least one illustrated embodiment.

FIG. 22F is a side view of the printed circuit board of FIGS. 22A-22D, without the associated components coupled thereto of FIGS. 22A-22D, according to at least one illustrated embodiment.

FIG. 22G is a front view of the printed circuit board of FIGS. 22A-22D, without the associated components coupled thereto of FIGS. 22A-22D, according to at least one illustrated embodiment.

FIG. 22H is a front view of an alternative configuration of the printed circuit board of FIGS. 22A-22D, without the associated components coupled thereto of FIGS. 22A-22D, according to at least one illustrated embodiment.

FIG. 23 is a three-dimensional rendering in a perspective view of the delivery device to deliver a vapor, a cloud, or an aerosol comprising scent media of FIGS. 21A-21F, according to at least one illustrated embodiment.

FIG. 24 is a three-dimensional rendering in a perspective view of an alternative configuration for the delivery device to deliver a vapor, a cloud, or an aerosol comprising scent media of FIG. 23, according to at least one illustrated embodiment.

FIG. 25 is a three-dimensional rendering in a perspective view of another alternative configuration for the delivery device to deliver a vapor, a cloud, or an aerosol comprising scent media of FIG. 23, according to at least one illustrated embodiment.

FIG. 26 is a three-dimensional rendering in a perspective view of another alternative configuration for the delivery device to deliver a vapor, a cloud, or an aerosol comprising scent media of FIG. 23, according to at least one illustrated embodiment.

FIG. 27 is a three-dimensional rendering in a perspective view of another alternative configuration for the delivery device to deliver a vapor, a cloud, or an aerosol comprising scent media of FIG. 23, according to at least one illustrated embodiment.

FIG. 28 is a three-dimensional rendering in a perspective view of another alternative configuration for the delivery device to deliver a vapor, a cloud, or an aerosol comprising scent media of FIG. 23, according to at least one illustrated embodiment.

FIG. 29 is a three-dimensional rendering in a perspective view of another alternative configuration for the delivery device to deliver a vapor, a cloud, or an aerosol comprising scent media of FIG. 23, according to at least one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with microcontrollers, piezo-electric devices, Peltier devices, power supplies such as DC/DC converters, wireless radios (i.e., transmitters, receivers or transceivers), computing systems including client and server computing systems, and networks (e.g., cellular, packet switched), as well as other communications channels, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

In particular, described herein are new compositions, systems, methods, and articles of manufacture to advantageously delivery of one or more active substances to the nose via retro-nasal delivery. Such can be employed in the up- regulation and, or, down-regulation of human and other animal metabolism. Such can additionally or alternatively be employed to produce other beneficial physiological effects, for example decongestion. Rather than deliver active substances to the nose via the standard ortho-nasal route, the compositions, apparatus, methods and articles described herein advantageously deliver active substances to the nose via the retro-nasal route.

These active substances or compositions are advantageously formulated as or in readily-soluble water droplets. The readily-soluble water droplets have a median size range of approximately 2 microns to approximately 50, 20, or 10 microns. Thus, the readily-soluble water droplets are too large for significant penetration into the lungs, while being small enough to be carried into the nose.

The active substances can be dissolved, if water soluble, directly in the water droplets. The active substances can, for example if not water soluble, be encapsulated inside, or otherwise formulated as, micelles, micro-emulsions, emulsions, liposomes, nanoparticles or other kinds of colloids. These colloids do not have a size larger than 500 nm, and optimally 200 nm or smaller. The small size of these colloids permits the nebulization of the droplets without destroying the colloids or otherwise impeding (e.g., clogging) the nebulizer (e.g., ultrasound transducer).

The droplets are delivered as a cloud to the mouth, for instance by the act of sipping. Sipping can either involve simply placing one's lips in a cloud of droplets containing the active material and sipping, or involve taking the droplets into the mouth via a conduit (e.g., a straw). On sipping the cloud, the droplets are delivered to the mouth where the droplets are suspended in the air in the mouth, and settle by gravity. The vapor around the droplets can immediately bring active substance into the nose via a “chimney effect” of the nose, and the droplets will themselves waft into the nose and deposit there, delivering active substances in a form that quickly acts on or within active tissue and resists quick clearance. Notably when the active substances are in small colloidal (e.g., nanoparticle) form, the colloids will themselves tend to resist clearance whereas larger particulates than those encompassed by the compositions described here will tend to be cleared through mucocilliary action. The active-substance-loaded droplets described here are produced from a small reservoir of less than 100 ML, optimally less than 50 ML, and particularly optimally less than 25 ML.

The composition, apparatus, methods and articles described herein have various useful benefits. The delivery of active substances or compositions (e.g., odorants or flavorful molecules of some kind, or other more traditional therapeutics) that up- and down-regulate metabolism, can be achieved with less than 1 gram of ingested active substance or composition. That is, the approaches described herein can deliver active substances to olfactory and/or taste receptors, producing physiological benefit (e.g., up-regulating and, or down-regulating human metabolism), while delivering almost no active substance to the gastro-intestinal (GI) tract. Second, for the purposes of delivery solely to the nose, as in the delivery of decongestants for the common cold or other respiratory dysfunction, the approaches described herein can produce greater physiological effect per nominal dose delivered to the nose than any other approach known by applicants, as in a spray or respiration from the environment into the nose.

Other kinds of water aerosols that are delivered to the mouth include electronic cigarettes and a methodology known as “Le Whaf.” Electronic cigarettes nebulize material into the mouth however with particle sizes that are small enough to penetrate the lungs and via the act of respiration, not sipping. Le Whaf produces mean particles sizes that are larger than 50 microns, thus not optimally suited to penetration into the nose. Other kinds of retro-nasal delivery of active substances exist in the form of highly volatile or aromatic lozenges, or food and drink, as in chocolate cake or a cup of coffee. These latter all involve ingested material with nominal masses placed in the mouth of greater than 1 gram.

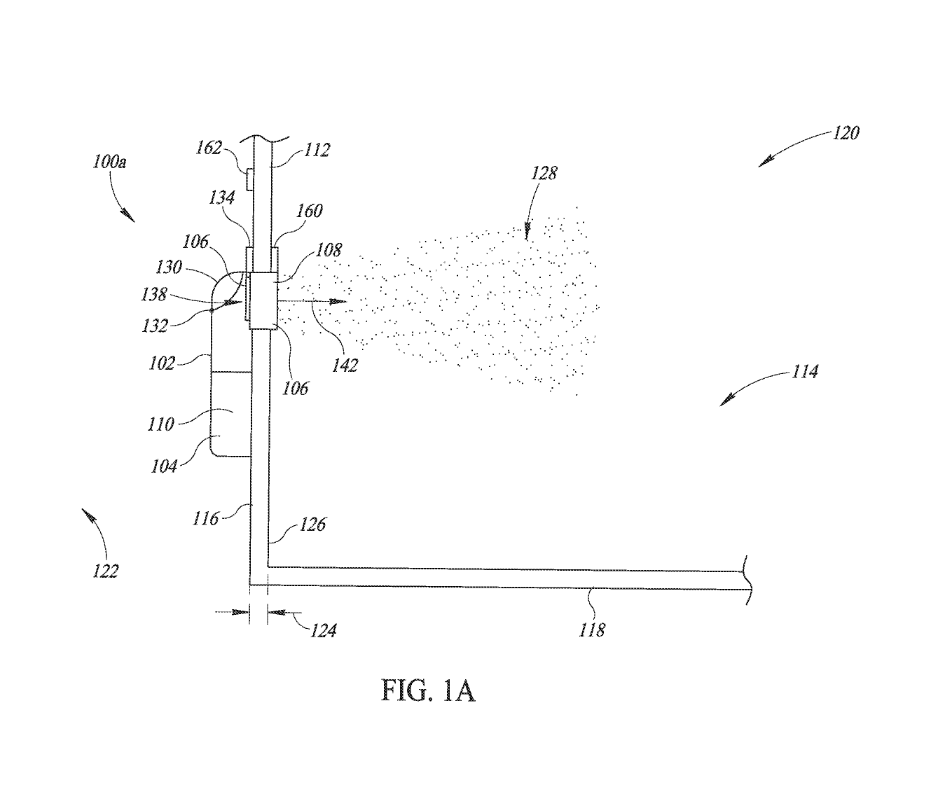

FIG. 1 shows a delivery system 100a that includes a housing 102, a actuator 106, and a fluidly communicative path 108, in which the delivery system 100a is coupled to a vessel 114. The vessel 114 may include at least one side wall (e.g., wall 112) and a bottom surface 118 that delineate an interior cavity or portion 120 of the vessel 114. As such, the vessel 114 may be used as a chamber to hold or dispense a cloud of aerosol or vaporized active substance within the interior portion 120. The side wall and bottom surface 118 may separate the interior portion 120 of the vessel 114 and an exterior portion 122. The wall 112 may have a thickness 124 that separates the exterior surface 116 from an interior surface 126. The vessel 114 may be comprised of a solid material, such as a hard plastic, ceramics, glass, or other similar material.

In some implementations, some or all of the delivery system 100a may be located proximate the exterior surface 116 of the wall 112 or other surface of a vessel 114. In some implementations, for example, an exterior surface 116 of the wall 112 may include a bracket, clasp, or other securing mechanism (not shown in FIG. 1A) that may be used to physically couple the housing 102 or other portion of the delivery system 100a to the vessel 114. In some implementations, the housing 102 may fluidly couple with the vessel 114 via one or more coupling or securing features via the fluidly communicative active substance path 108. In some implementations, some or all of the delivery system 100a may be located within the interior portion 120 of the vessel 114. For example, the housing 102 may be attached to the interior surface 126 and/or the bottom surface 118 of the vessel 114.

The housing 102 may include a fluid reservoir 104 and may be comprised of a polymer, elastomer, or other light-weight, durable material that may be used to hold a liquid. The housing 102 may be formed of one or more plastics, for example an ABS or polycarbonate plastic. The plastic may be injection molded or vacuum molded to form the housing 102. The type of material or process employed to form the housing 102 from the material should not be considered limiting. In some implementations, the housing 102 may include an interior cavity that forms the fluid reservoir 104 that may be used to hold and contain one or more active substances as a fluid 110 or other material (e.g., powder, gel, colloidal suspension) that carries active substances (e.g., scent molecules). In some implementations, for example, the fluid reservoir 104 may be sized and dimensioned to hold up to 100 mL of the fluid 110. In some implementations, the fluid reservoir 104 may be sized and dimensioned to hold a maximum amount of the fluid 110 that is less than 100 mL (e.g., 5 mL, 10 mL, 20 mL, 40 mL, or 50 mL). The fluid 110 may be any liquid or other material that is, or that carries, the active substance(s) (e.g., scent molecules) that are released when the fluid 110 transitions to a vapor or aerosol 128 and is released into the interior portion 120 of the vessel 114. The housing 102 may include an aperture 138 that forms part of the fluidly communicative active substance path 108 for the fluid 110 to be transferred from the fluid reservoir 104 to the interior portion 120 of the vessel 114 as the vapor or aerosol 128. The vapor or aerosol may advantageously comprise readily-soluble water droplets have a median size range of approximately 2 microns to approximately 50, 20, or 10 microns. Thus, the readily-soluble water droplets are too large for significant penetration into the lungs, while being small enough to be carried into the nose. The vapor or aerosol 128 may result in a physiological response from some users when those users encounter the active substance(s) transported by the vapor or aerosol 128.

In some implementations, the housing 102 may include a cover 130 or other covering that is selectively removable from an aperture that may be used to provide access to the fluid reservoir 104. In some implementations, the cover 130 may be pivotably coupled to the remaining portion of the housing 102 via one or more hinges 132 such that a user may selectively rotate the cover 130 along an axis of rotation formed by the one or more hinges 132. When opened, the cover 130 may provide access to the fluid reservoir 104 via a loading aperture 140 through which the fluid 110 may pass. Such fluid 110, for example, may be poured or dropped through the loading aperture 140 into the fluid reservoir 104.

The actuator 106 may be used to turn the fluid 110 stored in the fluid reservoir 104 into the vapor or aerosol 128 that is transmitted to the interior portion 120 of the vessel 114. In some implementations, for example, the actuator 106 is a nebulizer, for instance in the form of a transducer that oscillates a metal mesh to generate a mist, for example held or dispensed from in the interior portion 120 of the vessel 114. The transducer may oscillate at a frequency of about 175 kHz±5 kHz that is sufficient to atomize the fluid 110 held in the fluid reservoir 104. The frequency of oscillation of such a transducer may be increased or decreased depending up on the properties of the fluid 110 or other materials held within the fluid reservoir 104. In such an implementation, that transducer may form an annular ring with a metal-mesh included within a center portion of the transducer. As such, the fluid 110 may be transported to the metal mesh, via, for example, capillary action, where it is atomized into the vapor or aerosol 128 as a result of the oscillation of the transducer. In such an implementation, the actuator 106 may be located towards the bottom of the housing 102 such as to be in contact with the fluid 110.

In some implementations, the actuator 106 is electrically coupled to a battery 134 that may provide a power source for the oscillation of the scent actuator 106. The battery 134 may be small and lightweight, such as the batteries used for small electronic devices (e.g., hearing aids). In some implementations, the battery 134 is at least partially embedded within the thickness 124 of the wall 112. In some implementations, the battery 134 is selectively removable and replaceable, such as when the battery can no longer provide sufficient charge to operate the scent actuator 106. Other types of power sources may be provided, such as a power source comprised of one or more photovoltaic panels and associated components that may convert light into energy that can be used to operate the scent actuator 106, an array of super- or ultracapacitor cells, or an array of fuel cells.

The fluidly communicative active substance path 108 includes an opening or aperture through which atomized fluid particles can be introduced to the interior portion 120 of the vessel 114 as the vapor or aerosol 128. The opening for the fluidly communicative scent path 108 may extend across the thickness 124 of the wall 112 from the exterior surface 116 to the interior surface 126. In some implementations, the opening for the fluidly communicative active substance path 108 is substantially circular in shape, and is sized and dimensioned to securely hold the actuator 106. In some implementations, the fluidly communicative active substance path 108 includes an outer ring 136 between the actuator 106 and the wall 112. The outer ring 136 may be comprised of a compressible, elastic substance that, when compressed, forms a water-tight barrier between the reservoir 110 and the interior portion 120 of the vessel 114. As such, the compressible barrier may prevent any beverage or other liquid contained within the interior portion 120 of the vessel 114 from transferring to the fluid reservoir 104, as well as prevent any fluid 110 held within the fluid reservoir 104 from transferring to the interior portion 120 of the vessel 114 (except as the vapor or aerosol 128).

In some implementations, the fluidly communicative active substance path 108 may include a latch or other securing mechanism that may securely, physically couple to the housing 102. In some implementations, for example, the housing 102 may include a lip or raised portion that surrounds the aperture 138, with a corresponding groove being formed along an outer edge of the fluidly communicative active substance path 108. To couple the housing 102 to the fluidly communicative active substance path 108, the lip or raised portion on the housing 102 is inserted into and securely held by the corresponding groove included within the fluidly communicative s active substance cent path 108.

In some implementations, the fluidly communicative active substance path 108 may be used to position the actuator 106 and thereby direct the flow of the vapor or aerosol 128 upon entering the interior portion 120 of the vessel 114. As shown in FIG. 1, the opening of the fluidly communicative active substance path 108 may position the actuator 106 to direct the flow of the vapor or aerosol 128 substantially along a horizontal line 142 that may extend in a direction that is parallel, or substantially parallel, to the bottom surface 118 of the vessel 114. In some implementations, the fluidly communicative active substance path 108 may be positioned to direct the flow of the vapor or aerosol 128 in a direction that is above or below the horizontal line 142. For example, in some implementations, the fluidly communicative active substance path 108 may position the actuator 106 to direct the vapor or aerosol 128 along a line that is at an angle above the horizontal line 142. In some implementations, the fluidly communicative active substance path 108 positions the actuator 106 at an angle such that the vapor or aerosol 128 remains within the interior portion 120 of the open vessel 114 without escaping over the wall 112 to the exterior portion 122 of the vessel 114.

In some implementations a valve may be positioned within the fluidly communicative active substance path 108 that may be selectively opened or closed to enable the transmission of vapor or aerosol 128 into the interior portion 120 of the vessel 114. The valve may be actuated by an electrical signal that may be received via, for example, a control system 160 that may include a microcontroller. A suitable microcontroller may take the form of an 8-bit microcontroller with in-system programmable flash memory, such as the microcontroller commercially available from Atmel Corporation under designation ATMEGA48/88/168-AU. The microcontroller executes a program stored in its memory, and sends signals to control the various other components, such as, for example, the valves. Control signals may, for instance be pulse width modulated (PWM) control signal, particularly where controlling an active power supply device. Otherwise, control signals may take on any of a large variety of forms. For instance, the microcontroller may valves or the actuator 106 simply by completing a circuit that powers the respective value or actuator 106.

The delivery system 100a may optionally include a visual indicator 162 to indicate when the delivery system 100a is operating or turned ON. Although illustrated as a single light emitting diode (LED), the visual indicator 162 may take any of a large variety of forms. The LED may be capable of emitting one, two or more distinct colors. The visual indicator 162 may also indicate other information or conditions, for instance the visual indicator 162 may flash in response to an occurrence of an error condition. A pattern of flashes (e.g., number of sequential flashes, color of flashes, number and color of sequential flashes) may be used to indicate which of a number of possible error conditions has occurred.

FIG. 1B shows another version of the delivery system 100b that includes the housing 102, the actuator 106, and the fluidly communicative active substance path 108, in which the housing 102 is configured, sized, and shaped to receive a capsule 144 that contains the fluid 110. The capsule 144 may be comprised, for example, of a gel or plastic-like exterior that encloses an interior region that contains the fluid 110. The capsule 144 may be inserted into the fluid reservoir 104 selectively moving the cover 130 to an open position. The fluid reservoir 104 may include a protrusion 146 (e.g., point, blade, sharp edge) that may be used to puncture the exterior of the capsule 144 to thereby provide an exit for the fluid 110 from the interior of the capsule 144. In some implementations, a force may be applied against the capsule 144 when the cover 130 transitions from the open position to the closed position. This force may thereby press the capsule 144 against the protrusion 146 and cause at least a portion of the exterior of the capsule 144 to rupture, releasing the fluid 110 from the interior of the capsule. The fluid 110 may then be atomized by the actuator 106 and turned into the vapor or aerosol 128, as discussed above.

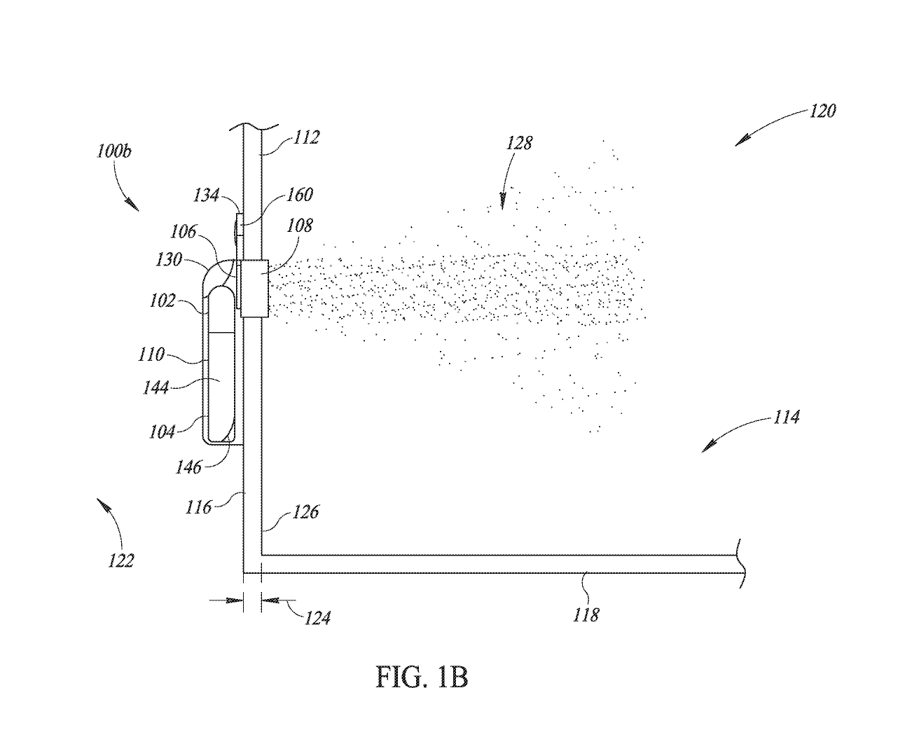

FIG. 2 shows delivery system 100 as viewed from an interior portion 120 of a vessel 114. As such, the housing 102 may be located on the opposite side of the wall 112 and as such, is provided in a dashed line representation. The fluidly communicative active substance path 108 may include a circular aperture 200 that extends through the thickness 124 of the wall 112 and may be delineated by an edge 202 of the wall 112 that surrounds the aperture 200. As previously noted, the fluidly communicative active substance path 108 may include the outer ring 136, which is positioned between the actuator 106 and the edge 202 of the wall 112. The actuator 106 may include a metal-mesh screen 204 that is used to atomize the fluid 110 into the vapor or aerosol 128. For example, in some implementations, the fluid 110 may be introduced to the metal-mesh screen 204 at a time during which the metal-mesh screen 204 is being vibrated or oscillated as part of the actuator 104. As the metal-mesh screen 204 is vibrated, the fluid 110 is atomized and transitions into the vapor or aerosol 128. In some implementations, the metal-mesh screen 242 may be fluidly coupled to the fluid reservoir 104 via capillaries, thereby providing a fluid path that enables a low flow of the fluid 110 from the fluid reservoir 104 to the metal-mesh screen 204. In some implementations, the metal-mesh screen 204 may provide a filter that prevents large sized molecules from being emitted as part of the vapor or aerosol 128 that enters the interior portion 120 of the vessel 114. As such, the metal-mesh screen 204 may have mesh openings 206 that are 500 micrometers in width. In some implementations, the mesh openings 206 may be less than 500 micrometers in width (e.g., 100 micrometers, 200 micrometers, 300 micrometers, or 400 micrometers). Preventing the larger molecules from being introduced into the interior portion 120 of the vessel 114 may provide for a better user experience by reducing the possibility that the vapor or aerosol 128 will irritate the user.

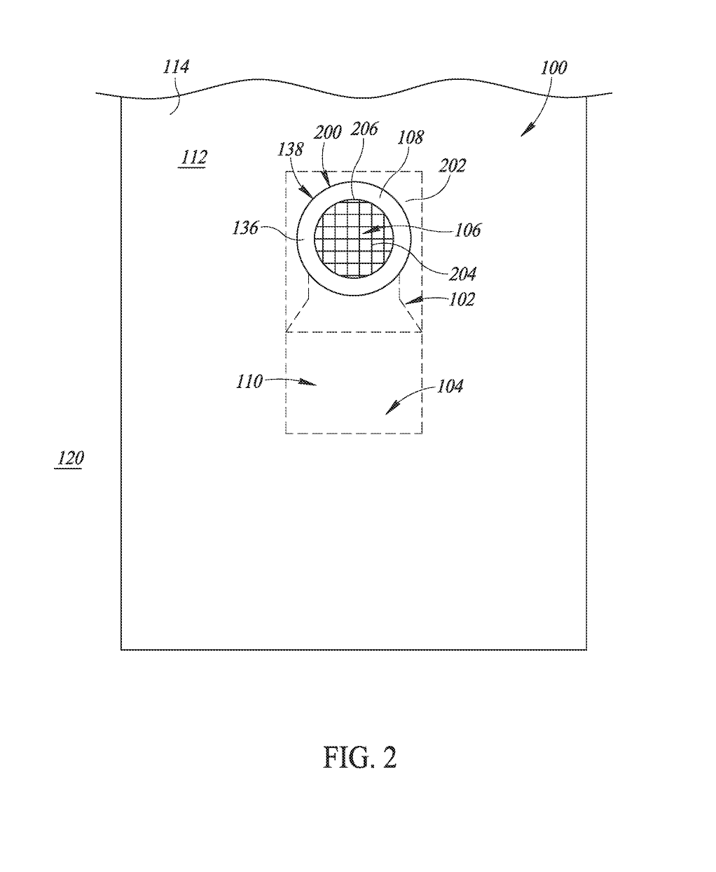

FIG. 3 shows delivery system 100 in which the aperture 138 to the fluidly communicative active substance path 108 directs a flow of the vapor or aerosol 128 from the actuator 104 at an angle 300 above the horizontal 302. In some implementations, the angle 300 may be, for example, 5°, 10°, 15°, 20°, 25°, 30°, 35°, 40°, 45°, or more above the horizontal 302. The angle 300 above the horizontal 302 may be chosen based upon, for example, the size of the vapor or aerosol molecules that the delivery system 100 produces, the position of an opening of the vessel 114 relative to the aperture 138 of the fluidly communicative active substance path 108, and a width of the opening of the vessel 114. In some implementations, the actuator 104 may be angled to direct the vapor or aerosol 128 into the interior portion 120 of the vessel 114 such that none, or only a small portion, of the vapor or aerosol 128 escapes from the interior portion 120 of the vessel 114.

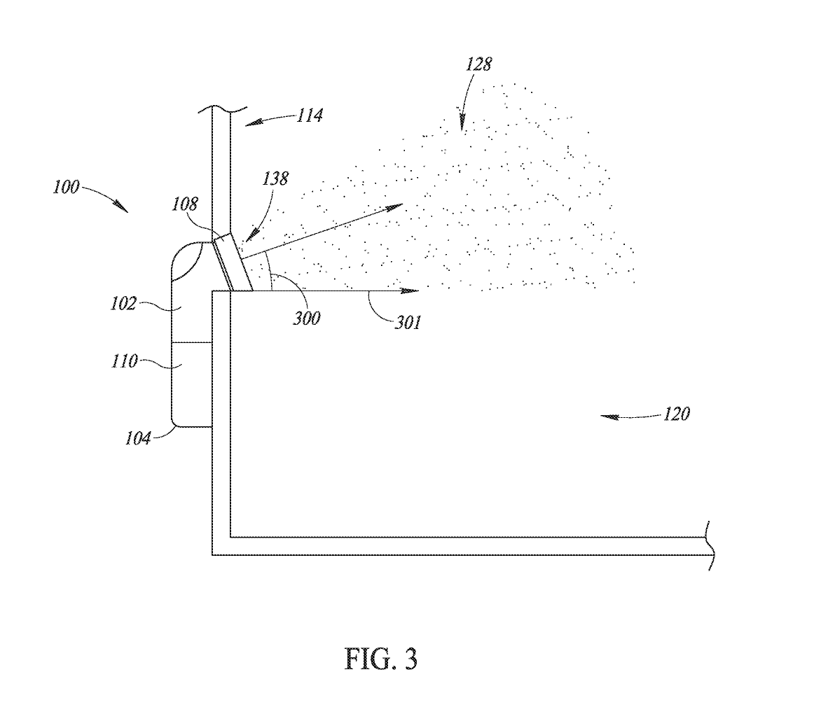

FIG. 4A and FIG. 4B show a delivery system 100 as viewed from an interior portion 120 of the vessel 114 in which a selectively removable cover 400 is rotatably coupled to the delivery system 100. The cover 400 may be coupled to the delivery system 100 via a pivot 402 that may provide an axis of rotation 404 positioned vertically, approximately parallel with the wall 112 of the vessel 114. In some implementations, the pivot 402 may be a hinge. In some implementations, the pivot 402 may be a flexible, bendable piece of plastic to enables the cover 400 to be rotated about the axis of rotation 404. As such, the cover 400 may be rotatable between an open position 450 (FIG. 4A) and a closed position 452 (FIG. 4A). In the closed position 452, the cover 400 may cover the aperture 138 of the fluidly communicative active substance path 108, thereby preventing vapor or aerosol 128 from being introduced into the interior portion 120 of the vessel 114. In some implementations, the cover 400 includes a latch or some similar securing feature that secures the cover 400 in the closed position 452. In the open position 450, the cover 400 may be located at a position removed from the aperture 138 of the fluidly communicative active substance path 108 to provide a path for the fluid 110 to be transferred to the interior portion 120 of the vessel 114 as the vapor or aerosol 128 via the actuator 104.

The cover 400 may be sized and shaped to completely cover the aperture 138 of the fluidly communicative active substance path 108 to the vessel 114 in the closed position 452. In some implementations, the cover 400 may have a first face 406 and a second face 407 that are separated by a width (not shown). The first face 406 may be substantially circular in shape with a diameter 408. The first face 406 may be positioned to face towards the inner portion 120 of the vessel 114 when the cover 400 is in the open position 450. The first face 406 may be rotated to face inwards, opposing the aperture 138 to the fluidly communicative active substance path 108 when the cover 400 is in the closed position 452. The first face 406 may be sized and shaped to be larger than the size of the aperture 138 to the fluidly communicative active substance path 108. In some implementations, for example, the diameter 408 of the cover 400 may be larger than a diameter 410 of the aperture 138 to the fluidly communicative active substance path 108 to thereby completely cover the aperture 138 to the fluidly communicative active substance path 108 when the cover 400 is in the closed position 452.

In some implementations, the cover 400 may include a latch or some similar securing feature that secures the cover 400 in the closed position 452. For example, the first face 406 of the cover 400 may include a latch or knob 412 that aligns with a corresponding notch or aperture 414 in or proximate to the interior surface 126 of the wall 112. The knob 412 may be secured within the aperture 414 thereby maintaining the cover 400 in the closed position 452. In some implementations, the first face 406 of the cover 400 may include a ridge or lip that extends circumferentially around an outside edge 416 of the first face 406 of the cover 400. The ridge may be sized and shaped to securely fit into a complementary depression that extends around or proximate to an outside portion of the aperture 138 of the fluidly communicative active substance path 108 along the interior surface 126 of the wall 112. The ridge extending around the first face 406 may be selectively, securely engaged with and secured within the depression running along, for example, the interior surface 126 of the wall 112 when the cover 400 is in the closed position 452. As such, the cover 400 may prevent the vapor or aerosol 128 from being introduced into the interior portion 120 of the vessel 114 by the actuator 108.

The cover 400 may include a cleaning surface 418 that extends across at least a portion of the first face 406 of the cover 400. The cleaning surface 418 may be sized and shaped to extend across all, or substantially all, of the metal-mesh screen 204 that is exposed within the aperture 138 of the fluidly communicative active substance path 108. The cleaning surface 418 may be fluidly coupled to a cleaning fluid reservoir 420 (FIG. 4B) that may be attached and located proximate the second face 407 of the cover 400. The cleaning fluid reservoir 420 may be used to hold a volume of cleaning fluid 422 (e.g., 5, 10, 15 mL or more). The cleaning fluid 422 may be, for example, any type of fluid that may be used to clean excess fluid 110 from the metal-mesh screen 204. Such a cleaning fluid 422 may be, for example, a solvent such as ethanol. The cleaning surface 418 may be comprised of a surface that may include, for example, a fabric surface that may draw or wick the cleaning fluid 422 from the cleaning reservoir to thereby wet or dampen the cleaning surface 418. When the cover 400 is rotated into the closed position 452, the cleaning surface 418 may be brought into contact with the metal-mesh screen 204 to thereby affect the cleaning of the metal-mesh screen 204 from excess fluid 110.

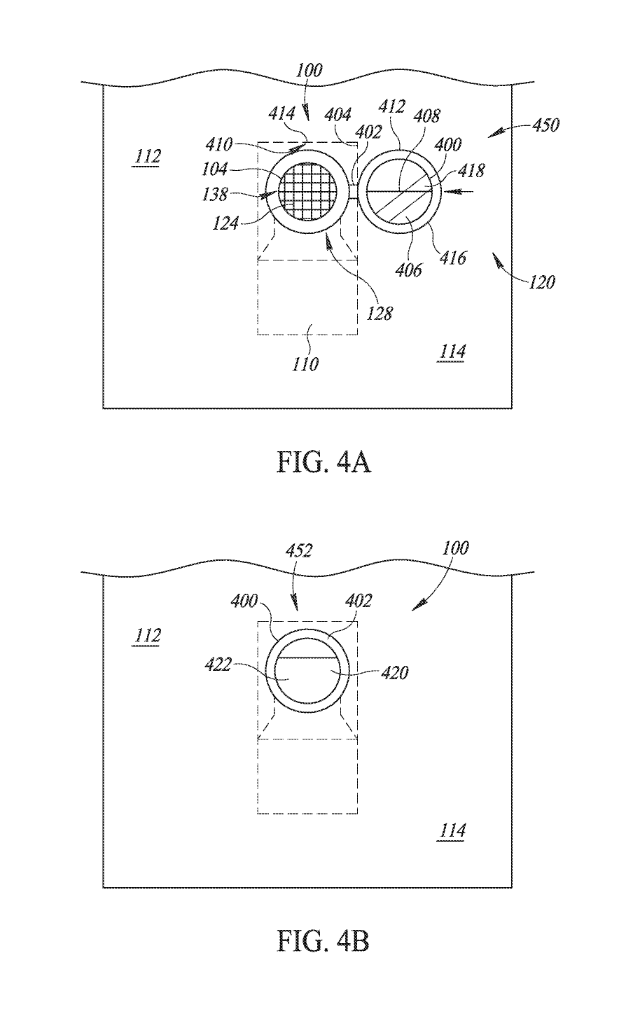

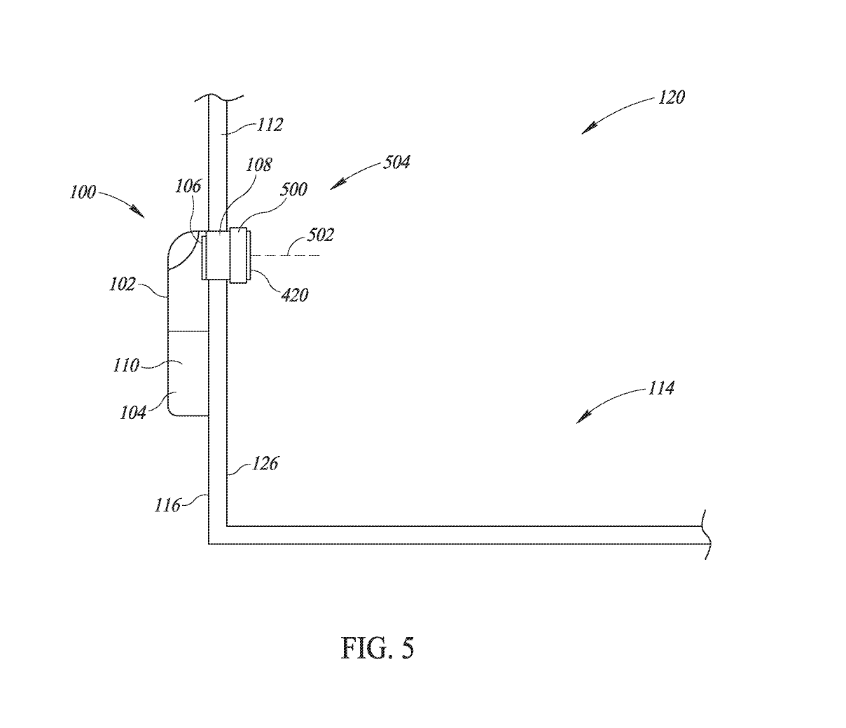

FIG. 5 and FIG. 6 show a twistable cover 500 that may be rotated about an axis of rotation 502 between a closed position 504 (FIG. 5) and an open position 600 (FIG. 6). The axis of rotation 502 may be perpendicular to the interior face 126 of the wall 112. In such an implementation, the twistable cover 500 may include a threaded, interior lip that engages with a corresponding threaded exterior lip along a portion of the fluidly communicative active substance path 108 that extends outward from the interior face 126 of the wall 112 towards the interior portion 120 of the vessel 114. In some implementations, the twistable cover 500 may include a cleaning surface 418 and associated cleaning fluid reservoir 420. The cleaning surface 418 may be located along an interior surface 604 of the twistable cover 500 such that the cleaning surface 418 opposes the metal-mesh screen 204 that may be included as part the delivery system 100 and located proximate the aperture 138 of the fluidly communicative active substance path 108.

The twistable cover 500 may be turned or twisted in a clockwise direction to transition the twistable cover 500 from the closed position 502 to the open position 600. Turning the twistable cover 500 in the clockwise direction may result a lateral motion of the twistable cover 500 away from the interior face 126, thereby exposing one or more openings 602 (FIG. 6) when the twistable cover 500 is in the open position 600. The openings 602 may provide a fluid path for the vapor or aerosol 128 to be introduced to the interior portion 120 of the vessel 112.

The twistable cover 500 may be turned or twisted in a counter-clockwise direction to transition the twistable cover 500 from the open position 600 to the closed position 502. In such a closed position 502, the twistable cover 500 may close off each of the openings 602, thereby preventing vapor or aerosol 128 from exiting the aperture 138 of the fluidly communicative scent path 108. When in the closed position 502, the twistable cover 500 may bring the cleaning surface 418 of the twistable cover 500 into contact with the metal-mesh screen 204. As such, the twistable cover 500 may be used to clean the metal-mesh screen 204 of debris or of residual scent fluid 110 when the twistable cover 500 is in the closed position 502.

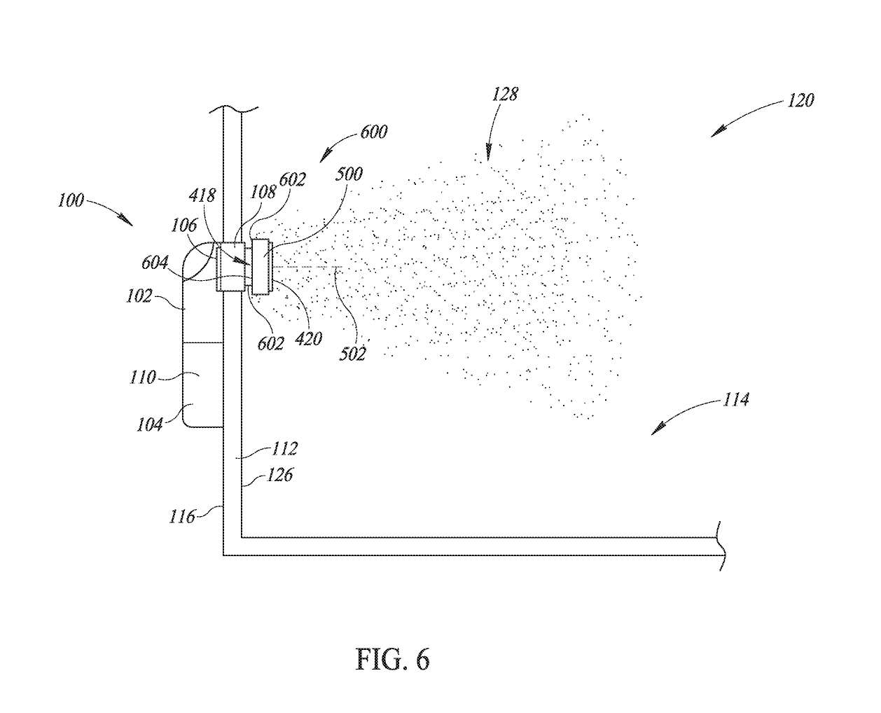

FIGS. 7A and 7B show an elongated container 700 as a vessel 114 in which the delivery system 100 is located within the interior portion 702 of the elongated container 700. Such an elongated container 700 may be shaped, for example, similarly to a cup or beverage container, and may be used to contain or hold the vapor or aerosol 128 for delivery to a user. The elongated container 700 may include a bottom portion 704 and a complementary top portion 706. The bottom portion 704 may be physically coupleable with the top portion 706. For example, in some implementations, the bottom portion 704 may include a threaded part along an upper rim 708, and the top portion 706 may include a complementary threaded part along a lower rim 710. The bottom portion 704 and the top portion 706 may be coupled together by engaging the complementary threaded parts. In some implementations, the bottom portion 704 may include one or more indentations that may couple with corresponding tabs on the top portion 706. In some implementations, one or both of the upper rim 708 of the bottom portion 704 and the lower rim 710 of the top portion 706 may include an elastic, compressible, annular layer that may provide an airtight/water tight seal when the bottom portion 704 and the top portion 706 are coupled.

The top portion 706 may include a neck 712 with one or more opening(s) 714. The opening(s) 714 may be used to draw vapor or aerosol 128 out of the interior portion 702 of the elongated container 700 by a user. The neck 712 may be tapered to concentrate the vapor or aerosol 128 as it is drawn upwards for consumption. The top portion 706 may be used to otherwise hold the vapor or aerosol within the elongated container 700 until the vapor or aerosol is consumed.

The bottom portion 704 of the elongated container 700 may have a substantially circular cross-section, and may be sized and shaped to be held and carried by a human user. The dispensing system 100 may be attached to an interior wall 716 and/or a floor 718 of the bottom portion 704 of the elongated container 700. In some implementations, some or all of the dispensing system 100 may form a unitary piece with the bottom portion 704 of the elongated container 700. For example, in some implementations, the dispensing system 100 may have a housing 720 that includes a front wall 722 that stretches across a portion of the interior wall 716. The front wall 722 may include one or more openings 726 through which vapor or aerosol 128 may be introduced to the interior portion 702 of the elongated container 700. The housing 720 may include a lid 723 that extends from the front wall 722 to the interior wall 716, thereby forming a cavity 724 within the interior portion 702 of the bottom portion 704. The lid 723 may be coupled to the front wall 722 and/or the interior wall 716 via, for example, one or more hinges 729, and be rotatable about an axis of rotation to thereby expose the cavity 724.

The cavity 724 may include a fluid reservoir 727 that may be used to hold the active substance(s) in a form of a fluid 110. The cavity 724 may include a holder or slot that is used to secure a scent actuator 106 proximate the opening(s) 714, between the actuator 106 and the fluid reservoir 727. The actuator 106 may be activated to introduce the vapor or aerosol 128 to the interior portion 702 of the elongated container 700. The reservoir 727 may be used to hold a quantity (e.g., (e.g., 5 mL, 10 mL, 20 mL, 40 mL, 50 mL, or up to 100 mL) of fluid 110 for use in the elongated container 700.

In some implementations, an elongated conduit (e.g., a straw) may be included to provide the user a way to draw the active substance into the user's mouth.

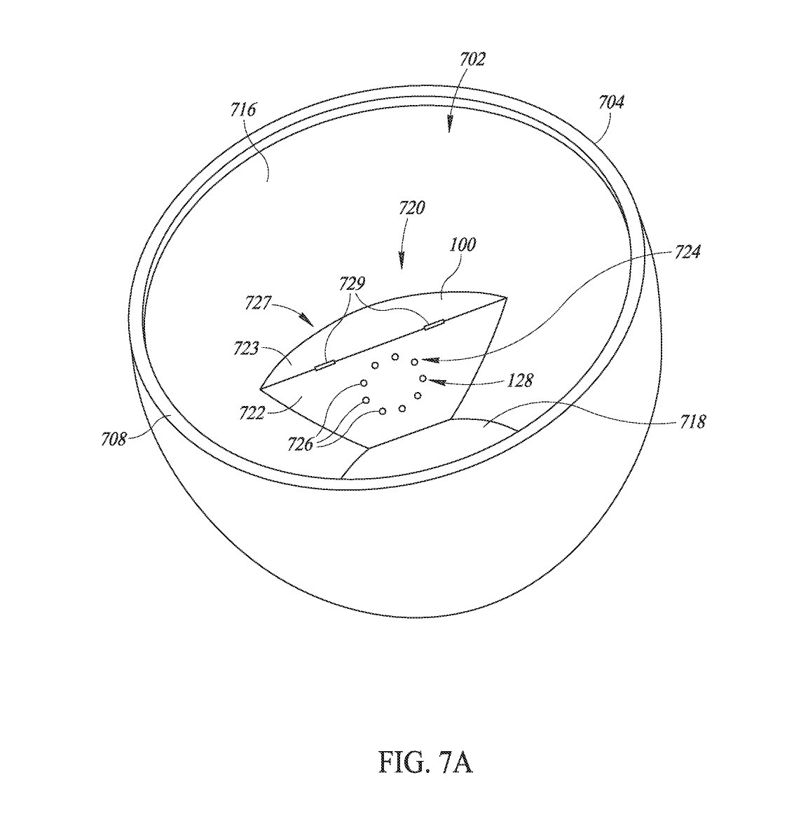

FIG. 8 shows a horizontal container 800 as a vessel 114 in which the delivery system 100 is located along an exterior wall 802 of the horizontal container 800. The delivery system 100 may be used to deliver a vapor or aerosol 128 to an area above the horizontal container 800. In such an implementation, the flow of the vapor or aerosol 128 may be in a direction that is substantially perpendicular to the direction 804 in which a mouth of a user is located.

In some implementations, a device is provided which takes the form of a vessel or at least includes a vessel portion, that in use holds a liquid. The device includes a scent media reservoir that is distinct from an interior or chamber of the vessel, and which in use holds scent media. The scent media is used to emit scent into the vessel, for example as a spray of droplets.

The vessel preferably takes the form of a glass, bowl, bottle, carafe, or even a spoon, and may have a shape that is customary to drink, eat or consume from so that the flavor clouds can shape common consumer food and beverage behavior.

The device may include a base, in addition to the vessel. The base may hold various electronics, for example a control subsystem, a nebulizer, etc.. Thus, the base can serve as a housing for electronics. The base can be made of any one or more of a large variety of materials, for example wood, metal or plastic. The base can be decorative in shape or configuration, or may bear decorative markings. The base can have any of a large variety of shapes, dimensions or configurations. For example, the base may take the form of a pedestal having a relatively large height or thickness. Also for example, the base may take the form of a coaster having a relative small height or thickness as compared to a width thereof. Also for example, the base may take the form of a tray having a relative small height or thickness as compared to a width thereof, either with or without a raised edge or rim. The base can have any of a large variety of profiles, for example circular, oval, square, rectangular, hexagonal, octagonal, or even freeform. The base can have a single area in which a vessel is locatable or removably receivable, or the base can have two or more areas in which respective ones of two or more vessels are locatable or removably receivable. The area or areas at which the vessel(s) can be removable received may have an associated switch or sensor that is responsive to a presence or absence of a vessel at the particular location, and which can produce a corresponding signal based on at least one of a presence or absence of a vessel at the respective location of the base.

The vessel may advantageously be removably supportable or even detachably coupleable to the base, allowing the vessel to be removed for cleaning, for example by placement into a dishwasher without presenting a danger to the electronics. The base may include attachment members to detachably couple to complimentary structures on the vessel, securing the vessel from inadvertently being knocked from the base. Attachment can be, for example, via a press fit, suction, adhesive, friction, magnets, clamps or the like.

The scent media reservoir may, for instance, hold a small amount of scent media in a liquid form. Suitable amounts can for example fall in the range of 2 milliliters to 10 milliliters inclusive. For instance, the scent media reservoir may hold 2 milliliters, 5 milliliters, or 10 milliliters of scent media in a liquid form. The small volume of the scent media reservoir assures not too much vapor or aerosol is placed into the air, which can be undesirable particularly if the liquid scent media is volatile. Use of a scent media reservoir that is separate from the vessel advantageously allows creation or generation of clouds of flavor/scent above food and drink, the clouds which differ from the aroma of the drink or food itself, and can be useful for conditioning appetite.

The scent media reservoir or outlet (e.g., port, nozzle) thereof may be positioned and oriented so that a spray or other distribution of scent media is directed towards, and optionally against, the inner surface (e.g., side wall) of the vessel. For example, a centerline of the spray intersects with an inner wall of the vessel. That is, a spray expands around a line of center of mass, and this centerline points to the inner wall. This advantageously ensures that the dispensed scent at least initially stays in the interior of the vessel, for instance until sampled or “ingested” by a human end user. The scent media reservoir may receive scent media in a liquid form. The scent media may, for example, be dripped or poured into the scent media reservoir. Alternatively the scent media may be contained in one or more cartridges that are removably, replaceably receivable by the scent media reservoir.

The vessel may have an opening, typically located at or at least proximate a top of the vessel, via which the vessel and optionally the scent media reservoir can be loaded, and from which the user may sample or “ingest” the contents of the vessel. The opening may advantageously be spaced above an outlet or nozzle from which the scent is dispensed or dispersed. Alternatively, or additionally, the opening or nozzle may be oriented to direct the spray of scent media away from the opening, for example at an angle to a major or rotational axis of the vessel, for instance in direction that is generally downward and pointed toward an interior surface of a side wall of the vessel. Again, such advantageously ensures that the dispensed scent at least initially stays in the interior of the vessel, for instance until sampled or “ingested” by a human end user.

In some implementations, the device may include a cover, the cover removably securable to the vessel, for example to selectively alternatingly provide and deny access to an interior of the vessel from an exterior thereof. In some implementations, the device may include an elongated hollow conduit (e.g., a straw), that provides a fluidly communicative path from an interior to the vessel to an exterior thereof, and which allows for a user or consumer to “sip out” or imbibe the “cloud” which is otherwise contained in the interior of the vessel. The use of a top and a straw advantageously allows a user to experience the flavor clouds and control the dose that is delivered, avoiding arbitrary escape from the vessel and assure metered delivery. This can be important for health and wellness applications.

The device may include a nebulizer, for example a nebulizer comprising a screen and a piezo-electric element, solenoid, or an electric motor physically (e.g., mechanically, magnetically) coupled to move (e.g., oscillate, rotate) the screen and thereby cause dispersion of the scent media in the interior of the vessel, for instance as a spray. The nebulizer may, for example, oscillate the screen at ultrasonic frequencies to cause a dispersion of the scent media. The nebulizer may be positioned spaced relatively below the opening of the vessel. For example, the nebulizer can be positioned spaced below the opening of the vessel and oriented to disperse scent media along a dispersion axis which is directed perpendicularly with respect to a portion of an interior surface of a side wall of the vessel. Alternatively, the nebulizer can be positioned spaced below the opening of the vessel and oriented to disperse scent media along a dispersion axis which is directed generally toward a portion of an interior surface of a side wall of the vessel, and which is also directed at a non-zero angle with respect to a perpendicular axis from the portion of the side wall toward the nebulizer, so as to point relatively downward with respect to a top or opening of the vessel. An orientation that causes a generally downward dispersion of the scent media may, for example, allow the nebulizer to advantageously be positioned closer to the top or opening of the vessel, while still ensuring that the dispersion of scent media is substantially directed and, or at least initially retained, within the interior of the vessel. Such positioning can enhance delivery to a user.

The device may include one or more switches or sensors communicatively coupled to operate the nebulizer. The device may, for example, include a switch that is operable from an exterior of the device, for instance a contact switch, a momentary contact switch, a rocker switch, etc. In at least one implementation, the switch may be mechanically coupled to a base or other portion of the device or vessel, and activated manually, e.g., by twisting the base or some part of the vessel. The device may, for example, include one or more sensors, for instance a one-, two- or three-axis accelerometer, a PIR motion sensor, an inductive sensor, a capacitive sensor, and, or Reed switches. The sensor(s) can detect a motion (e.g., upward movement, downward movement) and, or orientation (e.g., tilting, upright) of the device or vessel, and operate the nebulizer in response to some defined motion or orientation. The sensor(s) may detect a touch by the user or other contact (e.g., contact or absence of contact between a base of the device or vessel and some object, for instance a table) and operate the nebulizer in response to some defined touch or contact. The sensor(s) may detect a twisting of a vessel relative to a base, or even the placement of the vessel on the base, and operate the nebulizer in response. The sensor(s) may detect a presence or absence, for example a presence or absence of the vessel with respect to the base or a defined location on the base, and operate the nebulizer in response to some defined presence or absence. For instance, the base or the vessel may include one or more magnets, and the other one of the base or vessel may include a sensor (e.g., a Reed switch) that is response to the presence or absence of a magnet and associated magnetic field in the proximity of the sensor. In at least one implementation, placement of the vessel on the base is detected by the sensor, which provides a signal that causes the nebulizer to begin dispensing or dispersing scent in the interior of the vessel. Removal of the vessel from the base is also detected by the sensor, which in response provides a signal that causes the nebulizer to cease dispensing or dispersing scent in the interior of the vessel.

The device may include a transducer communicatively coupled to operate the nebulizer. The transducer may, for example, include one or more radios (e.g., cellular transceiver, WI-FI transceiver, Bluetooth transceiver) which receives wireless signals for instance RF or microwave signals for one or more wireless communications devices (e.g., smartphones) or remote controllers. The transducer may, for example, include one or more receivers, for instance an infrared receiver that receivers infrared light signals from a remote controller.

Activation may be synchronized with the delivery of audio, video, or audiovisual media. For example, a smartphone or digital assistance (e.g., Amazon Alexa®, Google Home®, Apple HomePod®) can cause activation of flavorful droplets inside a vessel that a consumer can experience in coordination with the delivery or experience of other digital media, e.g., music, film, video games, virtual reality (VR), augmented reality (AR), etc.

It may be desirable to create food and beverage experiences where the aroma associated with the food or beverage is enhanced, or other than it naturally would otherwise be. In this respect, flavor shapes appetite and cravings. Human appetites and cravings are largely shaped by flavor images that form in the brain while eating and drinking. It is possible to generate these images via delivery of flavorful droplets of minor mass, thereby shaping appetite and cravings without ingestion. Shaping the flavors, independently of the food and beverages themselves, makes it possible to help people eat and drink better and have more pleasurable experiences. This approach can be characterized as an “augmented reality” of scent and flavor, and the associated device denominated as an Augment Reality Vessel for Flavor Perception. The spray of droplets, aerosol or cloud of scent material may be characterized as a scent garnish. Droplets may have a media size of around 500 microns. These droplets may be delivered into open air within a vessel, preferably at angles that permit the droplets or cloud to remain principally contained inside the vessel. The approach can be used in entertainment (e.g., food or cooking programming), and in healthcare (e.g., control of metabolism).

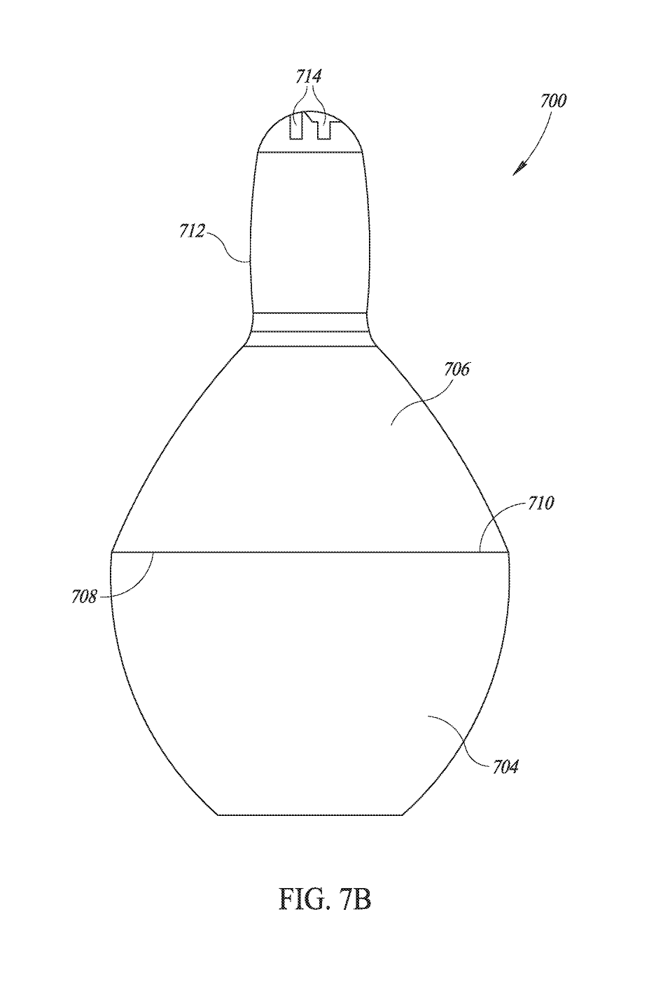

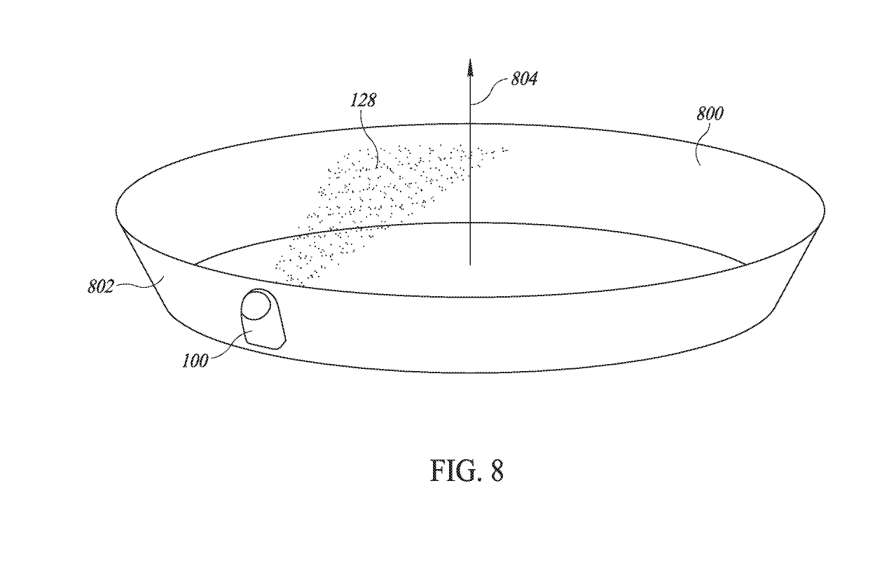

FIG. 9 is an isometric view of a device 900 including a vessel 902 with an open top 904, and a base 906 that includes electronics (FIG. 10) and on which the vessel 902 is removably or detachably supported, according to at least one illustrated implementation.

The vessel 902 may include an opening 908 via which scent media 910 is received, for instance as a spray pf droplets or an aerosol. The scent media 910 may be dispersed or dispensed via one or more components (e.g., nebulizer) contained in the base 906. The vessel may advantageously be removably coupleable to the base, allowing the vessel to be removed for cleaning, for example by placement into a dishwasher without presenting a danger to the electronics.

FIG. 10 is an isometric view of a portion of the device 900 of FIG. 9, including a nebulizer 1002, one or more actuators 1004, and a control subsystem 1006 and, or other electronics, according to at least one illustrated implementation.

The nebulizer 1002 can include one or more screens 1008 which is supported by a frame 1010 for movement, for example for oscillation or rotation The nebulizer 1002 can include one or more of a piezo-electric element 1012, solenoid 1014 or electric motor 1016 physically coupled to move the screen(s) 1008.

The actuators 1004 may include one or more of radios 1018, transducers or sensors 1020 and, or, switches 1022 communicatively coupled to the control subsystem 1006.

The control subsystem 1006 may, for example, include one or more microcontrollers 1024, microprocessors, field programmable gate arrays, and, or application specific integrated circuits. The control subsystem 1006 may, for example, include one or more nontransitory storage media 1026 that stores at least one of processor-executable instructions or data, which when executed by the microcontroller 1024 causes the microcontroller 1024 to control operation of the device 900, for example in response to one or more inputs. For example, the microcontroller may receive signals from one or more of radios 1018, transducers or sensors 1020 and, or, switches 1022, and control operation of the nebulizer 1002 in response to same. For instance, the control subsystem may cause the nebulizer to dispense or disperse scent media in response to a first input, and to stop the nebulizer from dispensing or dispersing scent media in response to a second input. Input can include user manipulation of a switch, positioning or orientation of the vessel by the user, or wireless commands from a radio or remote controller.

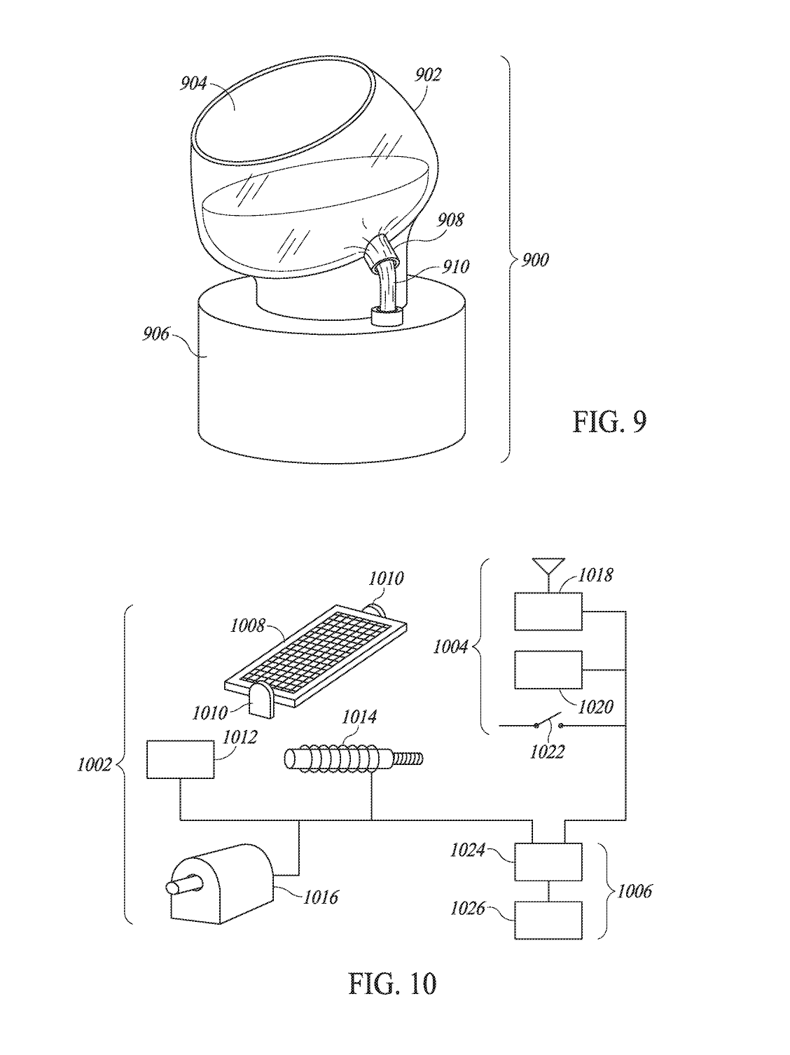

FIG. 11 is an isometric view of a portion of a device 1100 according to at least one illustrated implementation. The device 1100 includes a vessel 1102 with an open top 1104, at least one scent media reservoir 1106, and at least one nebulizer 1108. The nebulizer 1108 or a port 1110 thereof is positioned and oriented to dispense or disperse scent media in a direction that at least initially substantial retains the dispensed or dispersed scent media within an interior of the vessel 1102. For example, the nebulizer 1108 or a port 1110 thereof may be positioned relative below a rim that forms the open top. The nebulizer 1108 or a port 1110 thereof may be oriented to dispense or disperse scent media in a direction perpendicular to an inner surface or side wall 1112 of the vessel 1102, as illustrated by perpendicular arrow 1114. The nebulizer 1108 or a port 1110 thereof may be oriented to dispense or disperse scent media in a direction angled downward with respect to the top of the vessel 1102, as illustrated by arrow 1116. The nebulizer 1108 or a port 1110 thereof may be oriented to dispense or disperse scent media in a range that extends between perpendicular arrow 1114 and arrow 1116.

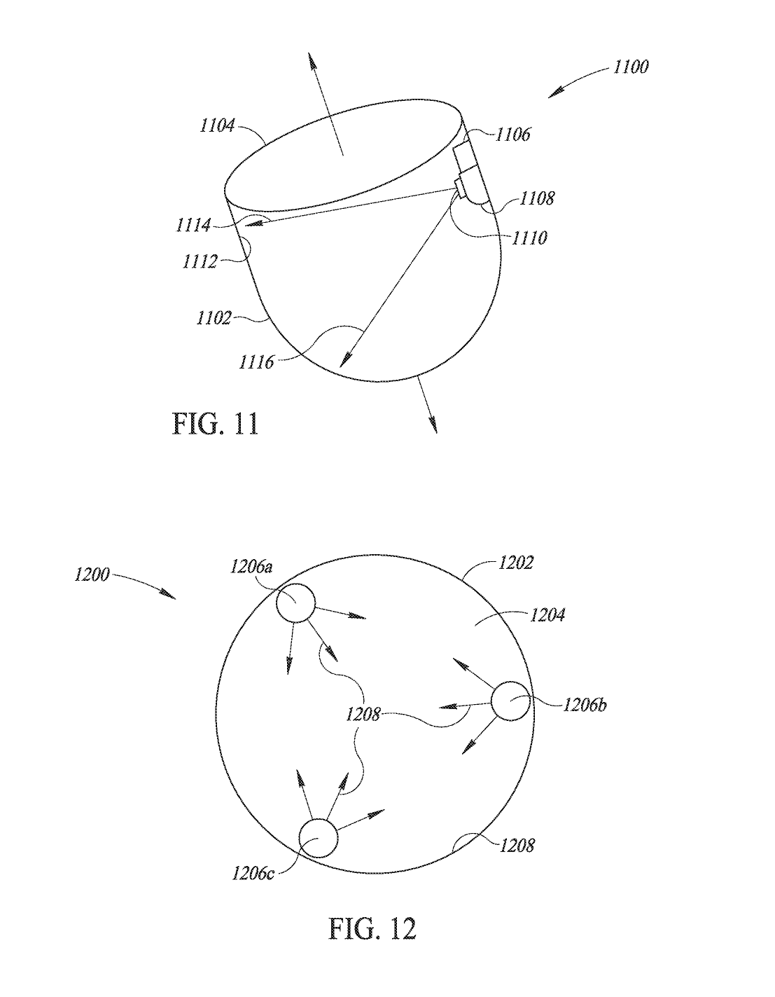

FIG. 12 is a top plan view of a portion of a device 1200, according to at least one illustrated implementation. The device 1200 includes a vessel 1202 with an open top 1204. The device 1200 includes three one scent media reservoirs and associated nebulizers (collectively 1206a, 1206b, 1206c) distributed about a periphery 1208 of the vessel 1202. While illustrated as radially spaced about the periphery in equal arc distances, the spacing between the reservoirs and associated nebulizers 1206a, 1206b, 1206c does not have to be equal. The nebulizers or a respective port thereof is positioned and oriented to dispense or disperse scent media (arrows 1208) in a direction that at least initially substantial retains the dispensed or dispersed scent media within an interior of the vessel 1202.

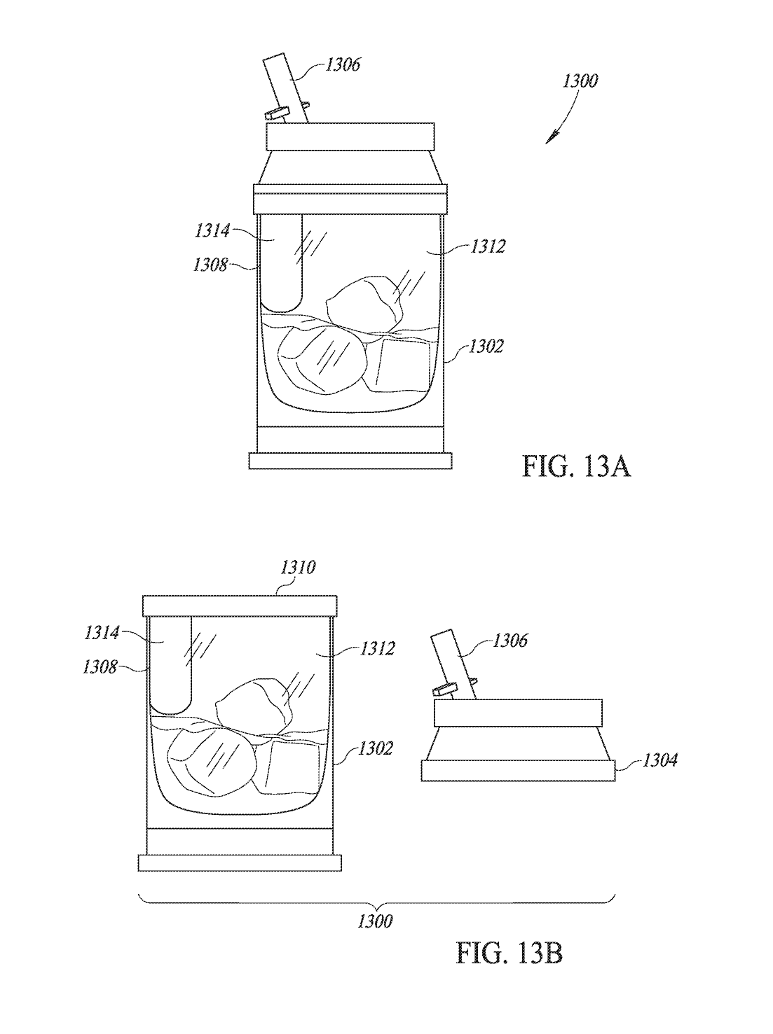

FIGS. 13A and 13B are side elevational views of a device 1300 including a vessel 1302 with a cover 1304, a conduit or straw 1306, and a scent media reservoir and associated nebulizer (collectively 1308), according to at least one illustrated implementation. In particular, FIG. 13A illustrates the cover 1304 is positioned to close a top or opening 1310 (FIG. 13B) of the vessel 1302, while FIG. 13B the cover 1304 removed from the vessel 1302 to open the top or opening 1310, thereby providing unhindered access to an interior 1312 of the vessel 1302. One or more cartridges 1314 may be removably retained via the scent media reservoirs.

In operation, the cover 1304 may be sufficient to ensure that a spray or aerosol dispersed or dispensed by the nebulizer at least initially remains in the interior 1312 of the vessel 1302. Alternatively or additionally, the nebulizer or a port thereof may optionally be positioned and oriented to dispense or disperse scent media in a direction that at least initially substantial retains the dispensed or dispersed scent media within an interior 1312 of the vessel 1302.

The vessel 1302 may contain a liquid (e.g., illustrated as a beverage) and, or a solid (e.g., food; illustrated as ice cubes). The scent media may be dispensed or dispersed (e.g., spray of droplets, aerosol) over the liquid and, or, solid which the user will imbibe, for example via a straw, the scent media changing or enhancing the liquid or solid which the user will imbibe.

The conduit or straw 1306 that provides a fluidly communicative path through the cover 1304 for a user to imbibe the contents of the vessel 1302 imbued with scent.

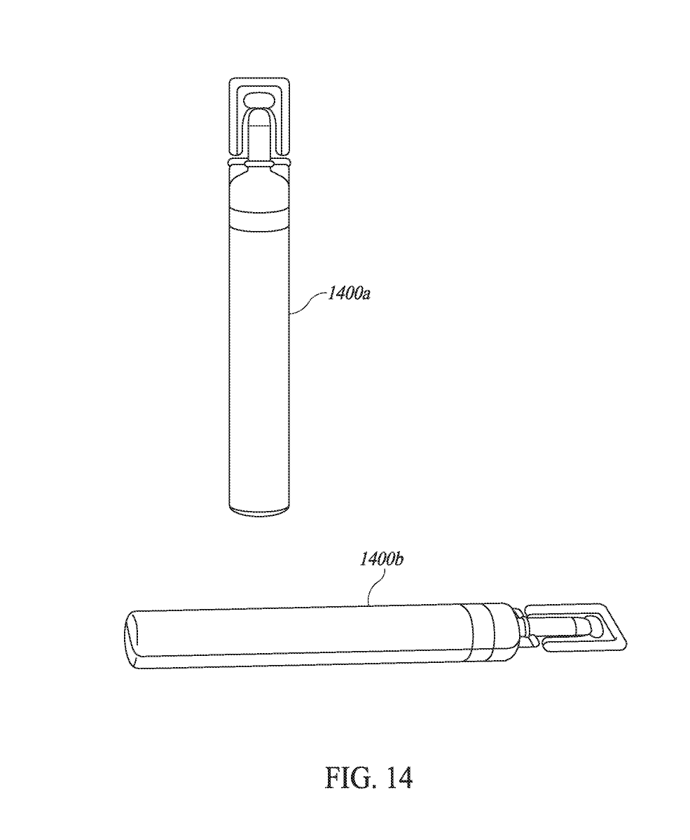

FIG. 14 is an isometric view of a pair of cartridges 1400a, 1440b that carry scent media, according to at least one illustrated implementation. The cartridges 1400a, 1440b are sized and dimensioned to be removably receivable by a scent media reservoir to supply scent media to the nebulizer for dispersion, for example as a spray of droplets or an aerosol. The cartridges 1400a, 1440b may be made of plastic. Single use cartridges 1400a, 1440b may, for example contain 2.5 milliliters of liquid.

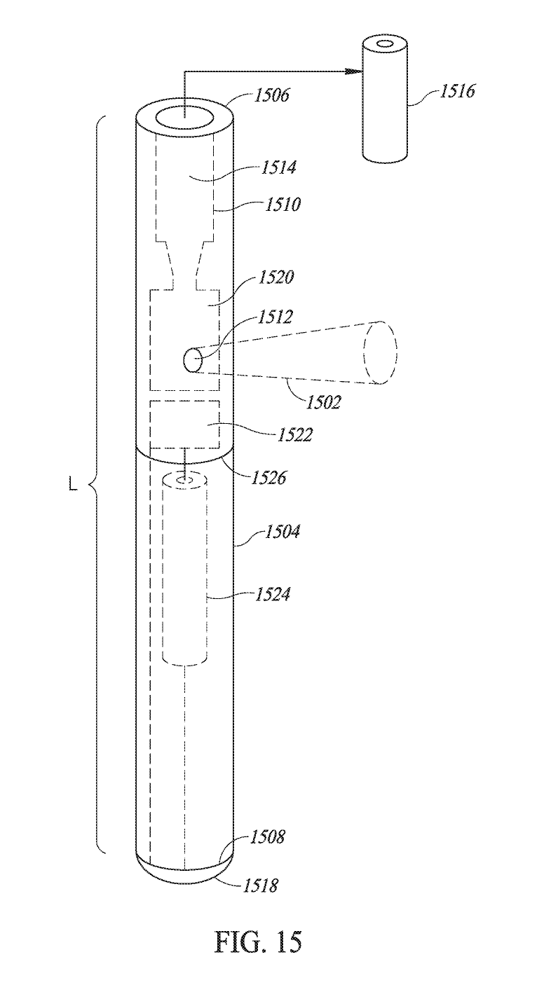

FIG. 15 is an isometric view of a delivery device 1500 to deliver a vapor or aerosol 1502 comprising active scent media over the contents of vessel, according to at least one illustrated implementation, illustrated along with a scent media cartridge.

The delivery device 1500 includes a housing or conduit 1504. The housing or conduit 1504 may be elongated, having a first or proximal end 1506 and a second or distal end 1508. The second or distal end 1508 may be opposed to the first or proximal end 1506 across a major dimension or length L of the delivery device 1500. The housing or conduit 1504 make resemble or take the form a stirrer, straw, or tube, and has a passage 1510 defined therethrough along at least a portion of the length L. The housing or conduit 1504 may include one or more outlet ports 1512 (only one illustrated). The outlet port(s) 1512 provide a path for dispersion of the vapor or aerosol 1502 from the delivery device 1500. The outlet port(s) 1512 are preferably positioned relatively closer to the first or proximate end 1506 than the second or distal end 1508, to position the outlet port(s) 1512 to be above a level of the food or beverage in the vessel, and preferably at an angle that dispenses at most perpendicular to the upper most level of the food or beverage, or even downward toward such. In some implementations, the outlet port(s) 1512 may be approximately (i.e., 15%) ⅓ down the length L of the housing or conduit 1504 from the first or proximal end 1506.

In some implementations, the housing or conduit 1504 can take the form of a circular cylinder, for example as illustrated in FIG. 15. In other implementations, the housing or conduit 1504 may have a non-circular cross-section, for example oval, rectangular, hexagonal, octagonal, etc., or may have a profile that varies along a length of the housing or conduit 1504. While illustrated as a straight elongated body, the housing or conduit 1504 does not necessarily need to be straight, and can have one or more curves or may even be a free-form body.

As illustrated in FIG. 15, the housing or conduit 1504 may have a scent media receptacle 1514. The scent media receptacle 1514 may, for example, be located at or proximate the first or proximal end 1506. The scent media receptacle 1514 is sized to receive scent media, either directly or contained in a scent media cartridge 1516 (shown removed).

As illustrated in FIG. 15, the housing or conduit 1504 may have a switch 1518 communicatively coupled to provide control signals in response to activation of the switch 1518. The switch 1518 may, for example, be located at or proximate the second or distal end 1508. This allows the switch 1518 to be activated by contact with a portion of a vessel into which the delivery device 1500 is at least partially inserted, For example, the delivery device 1500 may be at least partially inserted into a vessel that contains a food or beverage. A user may hold the delivery device 1500 by the proximate the first or proximal end 1506 while the second or distal end 1506 is positioned below an upper level of the food or beverage in the vessel. The user may bring the switch 1518 in contact with a portion (e.g., bottom, side wall) of the vessel, causing a signal to be generated to start dispensing a vapor or aerosol across the food or beverage in the vessel. The switch 1518 can take any of a large variety of forms, for example a contact switch, a momentary contact switch, a dome switch. In this implementation, the switch 1518 itself is preferably hermetically sealed from contact with the food or beverage, for example via a silicone dome or cover. Other suitable switches 1518 can include proximity switches or IR transceivers.

The delivery device 1500 includes a transducer, for example a nebulizer 1520 that is operable to cause formation of a vapor or aerosol 1502 comprising readily-soluble droplets comprising the one or more active substances from the active substance media and to dispense the vapor or aerosol 1502 via the at least one outlet port 1512. The vapor or aerosol 1502 may advantageously take the form of readily-soluble droplets have a median size range of approximately 2 microns to approximately 50, 20, or 10 microns comprising the one or more active substances. The passage 1510 provides a communicative between the scent media receptacle 1514 and the nebulizer 1520 to provide scent media to the nebulizer 1520 from the scent media receptacle 1514.

The delivery device 1500 optionally includes a control subsystem 1522. The control subsystem 1522 includes circuitry that operates the nebulizer to produce the vapor or aerosol. The control subsystem 1522 may be communicatively coupled to the switch, for example via one or more wires, fiber optics or via an IR transceiver. The control subsystem 1522 may include a timer, for example to operate the nebulizer 1520 for a defined time period in response to receiving a control signal from the switch 1518 on activation of the switch 1518. Such may be particularly useful for controlling a length of time, and hence and amount of active media dispensed. Such may be particularly useful where the switch 1518 is a momentary switch, which, for example, provides a single signal in response to a single activation. The control subsystem 1522 may prevent subsequent dispersal for a defined period of time, or may prevent such until the delivery device has been removed from the vessel and reinsert. The delivery device 1500 may include one or more inertial sensors or fluid sensor to detect whether or not the delivery device has been removed from the food or beverage prior to allowing a subsequent dispersal and provide signals indicative of such to the control subsystem 1522. The control subsystem 1522 may monitor a quantity of scent media residing the in the scent media reservoir 1514 or a pressure, for example via one or more pressure sensors, load cells or other sensors. The control subsystem 1522 may cause presentation of a message when the amount remaining scent media falls below a threshold level.

The delivery device 1500 includes one or more power sources 1524, for example one or more of: primary battery cells, secondary battery cells, super- or ultra-capacitor cells, and, or fuel cells. The power source 1524 may be rechargeable, for instance via a set of electrical contacts or inductive charger element.

The housing or conduit 1504 may include two separable parts, with a seam 1526 therebetween, for example to allow the power source to be replaced. The parts may be include threads or other coupling structures (not shown) allowing the parts to be screwed apart and screwed together. One or more gaskets (not shown) can provide hermetical sealing of the interior of the housing or conduit 1504 from an exterior thereof.

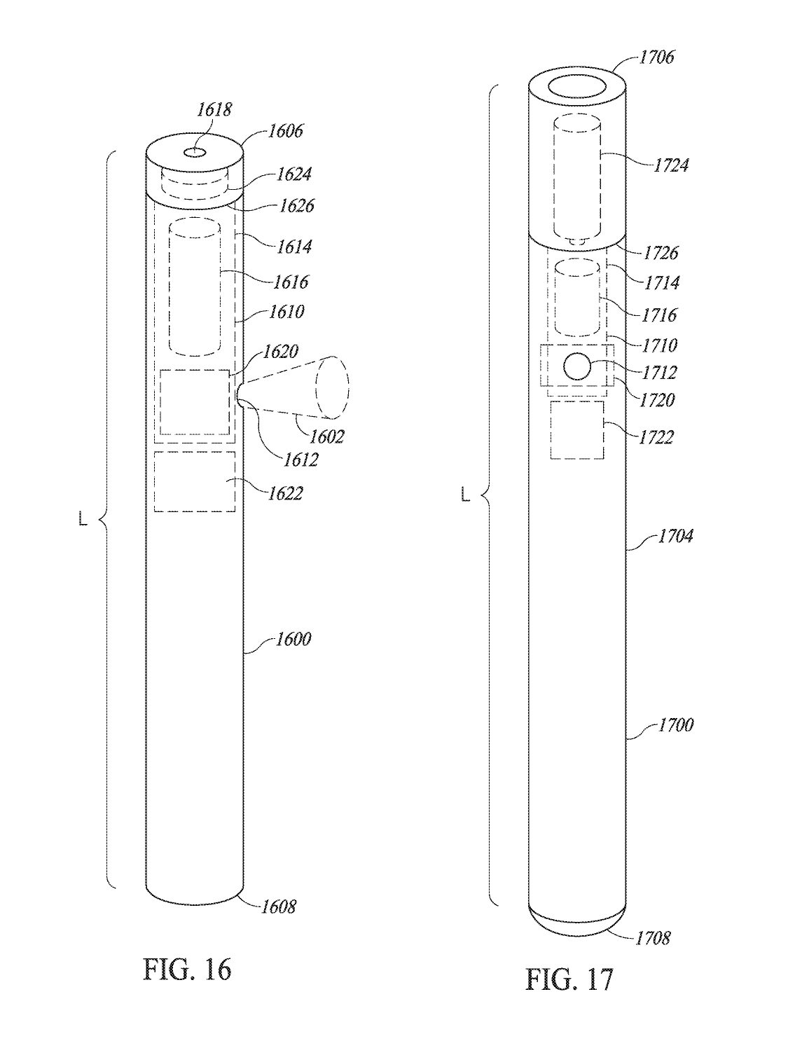

FIG. 16 is an isometric view of a delivery device 1600 to deliver a vapor or aerosol 1602 comprising active scent media over the contents of vessel, according to at least one illustrated implementation.

The delivery device 1600 includes a housing or conduit 1604. The housing or conduit 1604 may be elongated, having a first or proximal end 1606 and a second or distal end 1608. The second or distal end 1608 may be opposed to the first or proximal end 1606 across a major dimension or length L of the delivery device 1600. The housing or conduit 1604 make resemble or take the form a stirrer, straw, or tube, and has a passage 1610 defined therethrough along at least a portion of the length L. The housing or conduit 1604 may include one or more outlet ports 1512 (only one illustrated). The outlet port(s) 1612 provide a path for dispersion of the vapor or aerosol 1602 from the delivery device 1600. The outlet port(s) 1612 are preferably positioned relatively closer to the first or proximate end 1606 than the second or distal end 1608, to position the outlet port(s) 1612 to be above a level of the food or beverage in the vessel, and preferably at an angle that dispenses at most perpendicular to the upper most level of the food or beverage, or even downward toward such. In some implementations, the outlet port(s) 1612 may be approximately (i.e., 15%) ⅓ down the length L of the housing or conduit 1604 from the first or proximal end 1606.

In some implementations, the housing or conduit 1604 can take the form of a circular cylinder, for example as illustrated in FIG. 16. In other implementations, the housing or conduit 1604 may have a non-circular cross-section, for example oval, rectangular, hexagonal, octagonal, etc., or may have a profile that varies along a length of the housing or conduit 1604. While illustrated as a straight elongated body, the housing or conduit 1604 does not necessarily need to be straight, and can have one or more curves or may even be a free-form body.

As illustrated in FIG. 16, the housing or conduit 1604 may have a scent media receptacle 1614. The scent media receptacle 1614 may, for example, be located at or proximate the first or proximal end 1606. The scent media receptacle 1614 is sized to receive scent media, either directly or contained in a scent media cartridge 1616 (shown removed).

As illustrated in FIG. 16, the housing or conduit 1604 may have a switch 1618 communicatively coupled to provide control signals in response to activation of the switch 1618. The switch 1618 may, for example, be located at or proximate the first or proximal end 1606. This allows the switch 1618 to be activated by contact with a portion of a hand of a user with the second or distal end 1608 positioned in a vessel into which the delivery device 1600 is at least partially inserted, For example, the delivery device 1600 may be at least partially inserted into a vessel that contains a food or beverage. A user may hold the delivery device 1600 by the proximate the first or proximal end 1606 while the second or distal end 1606 is positioned below an upper level of the food or beverage in the vessel. The user may contact the switch 1618, causing a signal to be generated to start dispensing a vapor or aerosol across the food or beverage in the vessel. The switch 1618 can take any of a large variety of forms, for example a contact switch, a momentary contact switch, a dome switch. In this implementation, the switch 1618 itself is preferably hermetically sealed from contact with the user, for example via a silicone dome or cover. Other suitable switches 1618 can include proximity switches or IR transceivers.

The delivery device 1600 includes a transducer, for example a nebulizer 1620 that is operable to cause formation of a vapor or aerosol 1602 comprising readily-soluble droplets comprising the one or more active substances from the active substance media and to dispense the vapor or aerosol 1602 via the at least one outlet port 1612. The vapor or aerosol 1602 may advantageously take the form of readily-soluble droplets have a median size range of approximately 2 microns to approximately 50, 20, or 10 microns comprising the one or more active substances. The passage 1610 provides a communicative between the scent media receptacle 1614 and the nebulizer 1620 to provide scent media to the nebulizer 1620 from the scent media receptacle 1614.

The delivery device 1600 optionally includes a control subsystem 1622. The control subsystem 1622 includes circuitry that operates the nebulizer to produce the vapor or aerosol. The control subsystem 1622 may be communicatively coupled to the switch, for example via one or more wires, fiber optics or via an IR transceiver. The control subsystem 1622 may include a timer, for example to operate the nebulizer 1620 for a defined time period in response to receiving a control signal from the switch 1618 on activation of the switch 1618.

Such may be particularly useful for controlling a length of time, and hence and amount of active media dispensed. Such may be particularly useful where the switch 1618 is a momentary switch, which, for example, provides a single signal in response to a single activation. The control subsystem 1622 may prevent subsequent dispersal for a defined period of time, or may prevent such until the delivery device has been removed from the vessel and reinsert. The delivery device 1600 may include one or more inertial sensors or fluid sensor to detect whether or not the delivery device has been removed from the food or beverage prior to allowing a subsequent dispersal and provide signals indicative of such to the control subsystem 1622. The control subsystem 1622 may monitor a quantity of scent media residing the in the scent media reservoir 1614 or a pressure, for example via one or more pressure sensors, load cells or other sensors. The control subsystem 1622 may cause presentation of a message when the amount remaining scent media falls below a threshold level.

The delivery device 1600 includes one or more power sources 1624, for example one or more of: primary battery cells, secondary battery cells, super- or ultra-capacitor cells, and, or fuel cells. The power source 1624 may be rechargeable, for instance via a set of electrical contacts or inductive charger element.

The housing or conduit 1604 may include two separable parts, with a seam 1626 therebetween, for example to allow the power source to be replaced. The parts may be include threads or other coupling structures (not shown) allowing the parts to be screwed apart and screwed together. One or more gaskets (not shown) can provide hermetical sealing of the interior of the housing or conduit 1604 from an exterior thereof.

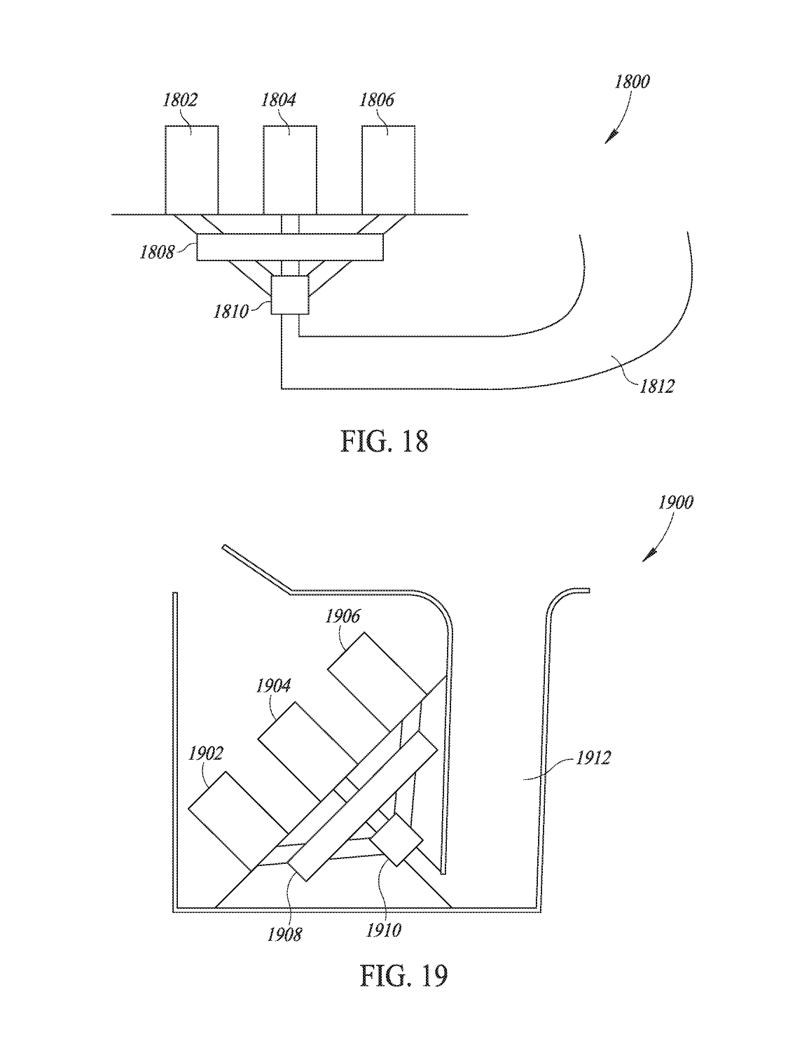

FIG. 17 is an isometric view of a delivery device 1700 to deliver a vapor or aerosol comprising active scent media over the contents of vessel, according to at least one illustrated implementation.

The delivery device 1700 includes a housing or conduit 1704. The housing or conduit 1704 may be elongated, having a first or proximal end 1706 and a second or distal end 1708. The second or distal end 1708 may be opposed to the first or proximal end 1706 across a major dimension or length L of the delivery device 1700. The housing or conduit 1704 make resemble or take the form a stirrer, straw, or tube, and has a passage 1710 defined therethrough along at least a portion of the length L. The housing or conduit 1704 may include one or more outlet ports 1612 (only one illustrated). The outlet port(s) 1712 provide a path for dispersion of the vapor or aerosol from the delivery device 1700. The outlet port(s) 1712 are preferably positioned relatively closer to the first or proximate end 1706 than the second or distal end 1708, to position the outlet port(s) 1712 to be above a level of the food or beverage in the vessel, and preferably at an angle that dispenses at most perpendicular to the upper most level of the food or beverage, or even downward toward such. In some implementations, the outlet port(s) 1712 may be approximately (i.e., 15%) ⅓ down the length L of the housing or conduit 1704 from the first or proximal end 1706.

In some implementations, the housing or conduit 1704 can take the form of a circular cylinder, for example as illustrated in FIG. 17. In other implementations, the housing or conduit 1704 may have a non-circular cross-section, for example oval, rectangular, hexagonal, octagonal, etc., or may have a profile that varies along a length of the housing or conduit 1704. While illustrated as a straight elongated body, the housing or conduit 1704 does not necessarily need to be straight, and can have one or more curves or may even be a free-form body.

As illustrated in FIG. 17, the housing or conduit 1704 may have a scent media receptacle 1714. The scent media receptacle 1714 may, for example, be located at or proximate the first or proximal end 1706. The scent media receptacle 1714 is sized to receive scent media, either directly or contained in a scent media cartridge 1716 (shown removed).

As illustrated in FIG. 17, the housing or conduit 1704 may have a switch 1718 communicatively coupled to provide control signals in response to activation of the switch 1718. The switch 1718 may, for example, be located at or proximate the second or distal end 1708. This allows the switch 1718 to be activated by contact with a portion of a hand of a user with the second or distal end 1708 positioned in a vessel into which the delivery device 1700 is at least partially inserted, for example, the delivery device 1700 may be at least partially inserted into a vessel that contains a food or beverage. A user may hold the delivery device 1700 by the proximate the first or proximal end 1606 while the second or distal end 1706 is positioned below an upper level of the food or beverage in the vessel. The user may contact the switch 1718, causing a signal to be generated to start dispensing a vapor or aerosol across the food or beverage in the vessel. The switch 1718 can take any of a large variety of forms, for example a contact switch, a momentary contact switch, a dome switch. In this implementation, the switch 1718 itself is preferably hermetically sealed from contact with the user, for example via a silicone dome or cover. Other suitable switches 1718 can include proximity switches or IR transceivers.

The delivery device 1700 includes a transducer, for example a nebulizer 1720 that is operable to cause formation of a vapor or aerosol comprising readily-soluble droplets comprising the one or more active substances from the active substance media and to dispense the vapor or aerosol via the at least one outlet port 1712. The vapor or aerosol 1702 may advantageously take the form of readily-soluble droplets have a median size range of approximately 2 microns to approximately 50, 20, or 10 microns comprising the one or more active substances. The passage 1710 provides a communicative between the scent media receptacle 1714 and the nebulizer 1720 to provide scent media to the nebulizer 1720 from the scent media receptacle 1714.

The delivery device 1700 optionally includes a control subsystem 1722. The control subsystem 1722 includes circuitry that operates the nebulizer to produce the vapor or aerosol. The control subsystem 1722 may be communicatively coupled to the switch, for example via one or more wires, fiber optics or via an IR transceiver. The control subsystem 1722 may include a timer, for example to operate the nebulizer 1720 for a defined time period in response to receiving a control signal from the switch 1718 on activation of the switch 1718. Such may be particularly useful for controlling a length of time, and hence and amount of active media dispensed. Such may be particularly useful where the switch 1718 is a momentary switch, which, for example, provides a single signal in response to a single activation. The control subsystem 1722 may prevent subsequent dispersal for a defined period of time, or may prevent such until the delivery device has been removed from the vessel and reinsert. The delivery device 1700 may include one or more inertial sensors or fluid sensor to detect whether or not the delivery device has been removed from the food or beverage prior to allowing a subsequent dispersal and provide signals indicative of such to the control subsystem 1722. The control subsystem 1722 may monitor a quantity of scent media residing the in the scent media reservoir 1714 or a pressure, for example via one or more pressure sensors, load cells or other sensors. The control subsystem 1722 may cause presentation of a message when the amount remaining scent media falls below a threshold level.

The delivery device 1700 includes one or more power sources 1724, for example one or more of: primary battery cells, secondary battery cells, super- or ultra-capacitor cells, and, or fuel cells. The power source 1724 may be rechargeable, for instance via a set of electrical contacts or inductive charger element.

The housing or conduit 1704 may include two separable parts, with a seam 1726 therebetween, for example to allow the power source to be replaced. The parts may include threads or other coupling structures (not shown) allowing the parts to be screwed apart and screwed together. One or more gaskets (not shown) can provide hermetical sealing of the interior of the housing or conduit 1704 from an exterior thereof.

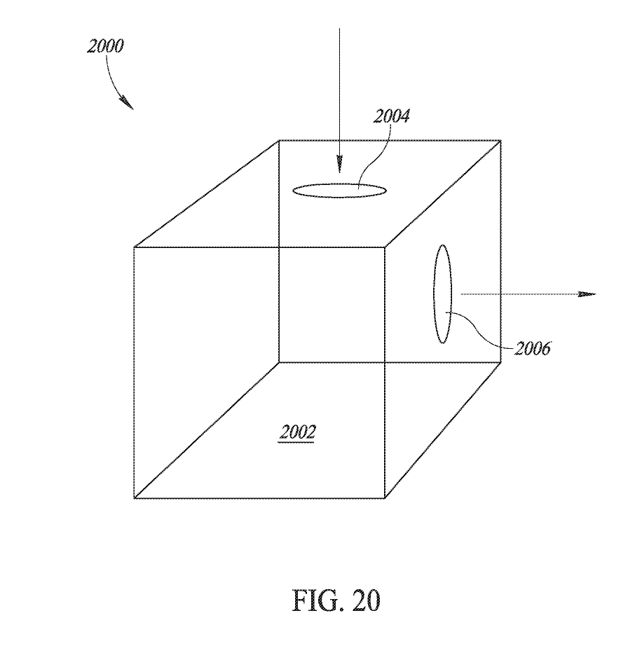

FIG. 18 illustrates a delivery device 1800 to deliver a vaporized scent media or a scent media in aerosol form. The delivery device 1800 can include any of the features of any of the other delivery devices described herein. Similarly, any of the other delivery devices described herein can include any of the features of the delivery device 1800. As shown in FIG. 18, the delivery device 1800 includes a first flavor vial 1802, a second flavor vial 1804, and a third flavor vial 1806. Each of the vials 1802, 1804, and 1806 can include a housing having any of the features of any of the other housings described herein, such as housing 102, and a fluid reservoir having any of the features of any of the other fluid reservoirs described herein, such as fluid reservoir 104. Each of the vials 1802, 1804, and 1806 can carry or hold a respective distinct scent media in liquid form, such as within the fluid reservoir thereof. For example, the vial 1802 can hold a first scent media in liquid form, the vial 1804 can hold a second scent media, which is distinct or different than the first scent media, in liquid form, and the vial 1806 can hold a third scent media, which is distinct or different than the first scent media and the second scent media, in liquid form.

FIG. 18 also illustrates that the delivery device 1800 includes an electronic orifice control unit 1808 and an ultrasound generator 1810. The orifice control unit 1808 can include three distinct valves, with each of the three valves controlling the flow of liquid out of a respective one of the vials 1802, 1804, and 1806 and toward the ultrasound generator 1810. The valves within the orifice control unit 1808 can have any of the features of any of the other valves described herein. The valves within the orifice control unit 1808 can be operable to open completely, so that liquid can flow therethrough at a maximum flow rate, close completely, so that liquid cannot flow therethrough, or open partially, so that liquid can flow therethrough at any desired fraction of the maximum flow rate. Thus, the orifice control unit 1808 and the valves thereof are operable to control the flow rate of liquid from each of the vials 1802, 1804, and 1806 to the ultrasound generator 1810, such as to control ratio(s) of the flowrates of the first scent media, the second scent media, and/or the third scent media to the ultrasound generator 1810.

FIG. 18 also illustrates that the delivery device 1800 includes a flavor cloud chamber 1812, through which a cloud of vaporized scent media or scent medial in aerosol form generated by the ultrasound generator 1810 can flow. The cloud of vaporized scent media or scent media in aerosol form can include a desired or controlled ratio of the first scent media to the second scent media, a desired or controlled ratio of the first scent media to the third scent media, and/or a desired or controlled ratio of the second scent media to the third scent media. A user of the delivery device 1800 can sip, imbibe, or otherwise consume the scent media directly from the flavor cloud chamber 1812, or the user can pour the scent media out of the flavor cloud chamber 1812, such as into another container or vessel for subsequent consumption. In some implementations, the delivery device 1800 produces the cloud of vaporized scent media or scent media in aerosol form with constant or relatively constant ratios between the first scent media, second scent media, and third scent media. In other implementations, the delivery device 1800 produces the cloud of vaporized scent media or scent media in aerosol form with ratios between the first scent media, second scent media, and third scent media that change, fluctuate, or oscillate over time.

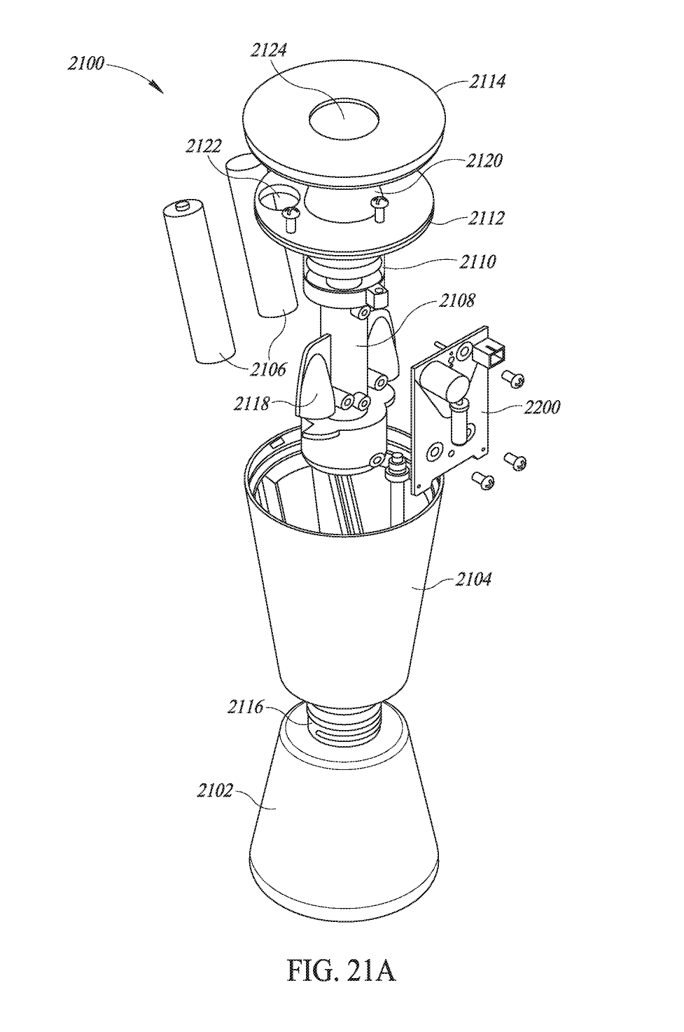

FIG. 19 illustrates another delivery device 1900 to deliver a vaporized scent media or a scent media in aerosol form. The delivery device 1900 can include any of the features described herein for delivery device 1800. For example, the delivery device 1900 includes a first flavor vial 1902 similar to the flavor vial 1802, a second flavor vial 1904 similar to the flavor vial 1804, and a third flavor vial 1906 similar to the flavor vial 1806. Further, the delivery device 1900 includes an electronic orifice control unit 1908 similar to the orifice control unit 1808, an ultrasound generator 1910 similar to the ultrasound generator 1810, and a flavor cloud chamber 1912 similar to the flavor cloud chamber 1812. As illustrated in FIG. 19, the vials 1902, 1904, and 1906, the orifice control unit 1908, and the ultrasound generator 1910, as well as the flow paths from the vials 1902, 1904, and 1906 to the orifice control unit 1908, the flow path from the orifice control unit 1908 to the ultrasound generator 1910, and the flow path from the ultrasound generator 1910 to the flavor cloud chamber 1912, are oriented downward, or such that gravity carries liquids or other materials along each of the flow paths toward the chamber 1912. Such orientations can assist in the operation of the delivery device 1900 by allowing the liquids or other materials to be carried through the system under their own weight by the effect of gravity.

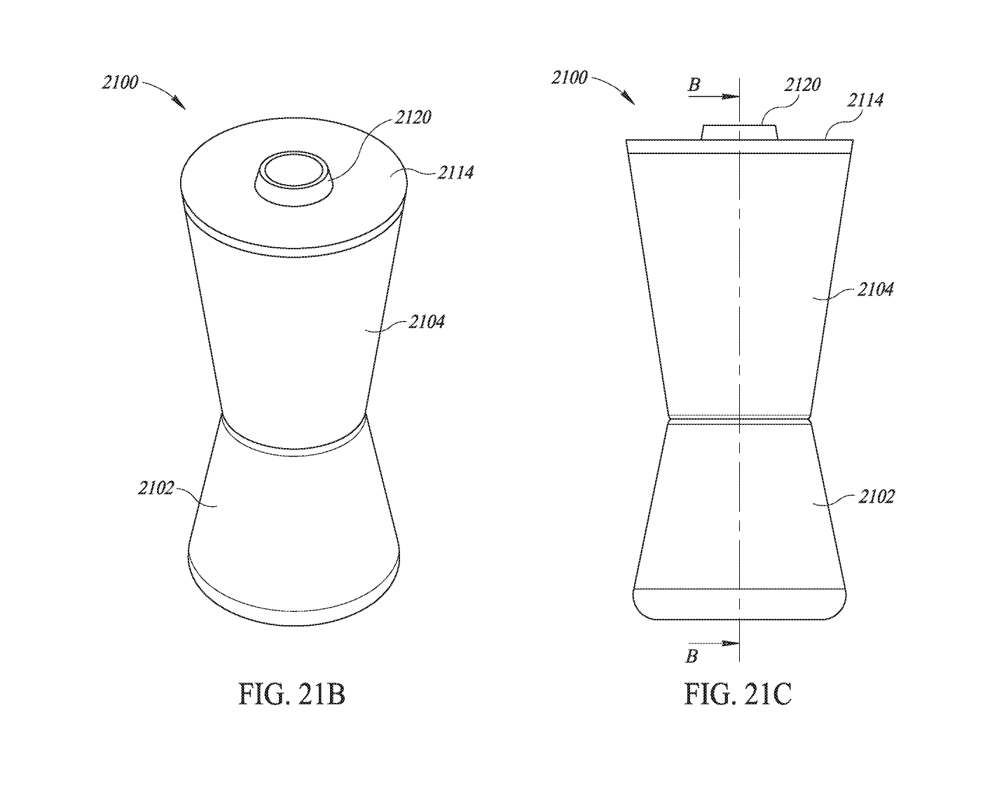

FIG. 20 illustrates an empty spacer device 2000 that can be used in combination with any of the scent media delivery devices described herein. As illustrated in FIG. 20, the spacer device 2000 includes an empty internal void or chamber 2002 and an inlet opening or hole 2004. An orifice of the scent media delivery device through which vaporized scent media or scent media in aerosol form is delivered is positioned at or within the inlet opening 2004 of the spacer device 2000, such that vaporized scent media or scent media in aerosol form generated by the delivery device exits the delivery device directly into the internal chamber 2002 of the spacer device 2000, thereby filling the internal chamber 2002 of the spacer device 2000 with a cloud of vaporized scent media or scent media in aerosol form.

A user can then remove the delivery device from the inlet opening 2004 and then sip, imbibe, or otherwise consume the vaporized scent media or scent media in aerosol form from the spacer device 2000 by positioning their mouth at the inlet opening 2004 and consuming the scent media through the inlet opening 2004. In one alternative implementation, the spacer device 2000 includes the inlet opening 2004 as well as an outlet opening or hole 2006, which may be provided with a mouthpiece. In such an implementation, once the internal chamber 2002 of the spacer device 2000 is filled with a cloud of vaporized scent media or scent media in aerosol form, the user can sip, imbibe, or otherwise consume the vaporized scent media or scent media in aerosol form from the spacer device 2000 by positioning their mouth at the outlet opening 2006 and consuming the scent media through the outlet opening 2006.

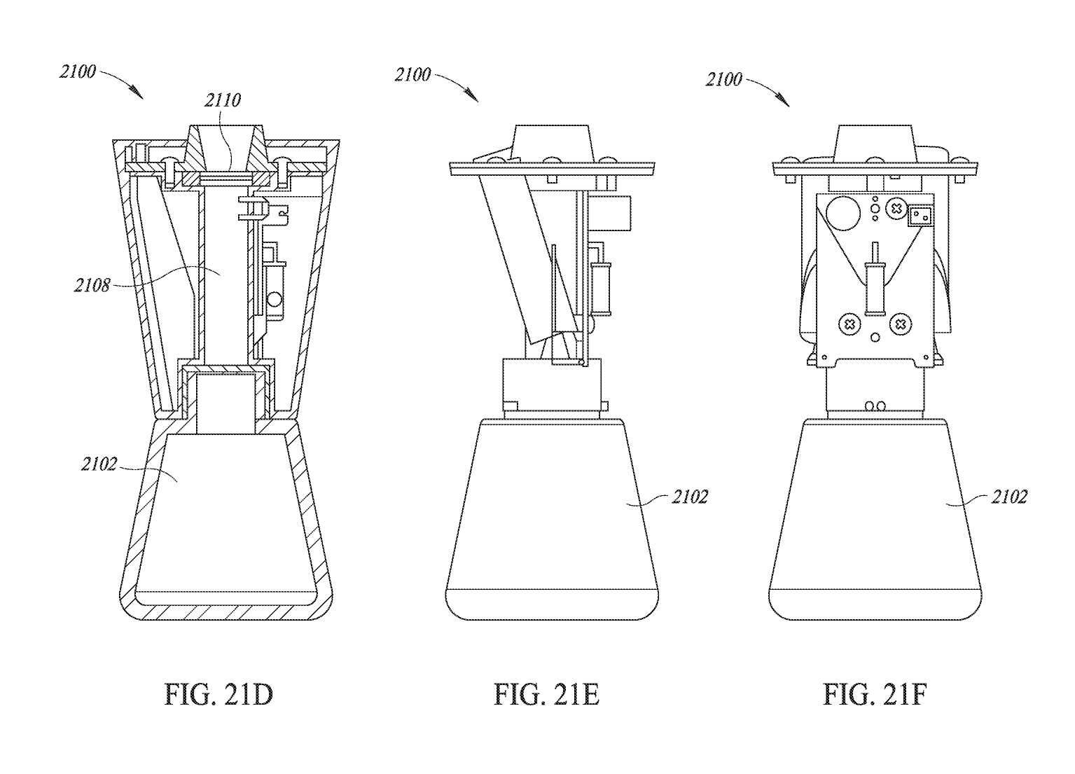

FIGS. 21A-21F illustrate various views of a handheld delivery device 2100 for producing and delivering a cloud of vaporized scent media or scent media in aerosol form. The device 2100 can include any of the features of any of the other devices described herein, such as the devices 1800 and 1900, and can be used in combination with any of the other devices described herein, such as the device 2000. As illustrated in FIG. 21A, delivery device 2100 includes a base 2102, which can be transparent and which includes a hollow container or tank or vial, in some cases having a volume or capacity of less than 100 mL, for holding scent media in a liquid form. The base 2102 also includes an upwardly-extending hollow conduit, tube, or pipe 2116, through which the scent media can be poured out of the base 2102 in a liquid form. An exterior surface of the conduit 2116 includes a set of threads.

The delivery device 2100 also includes a top or upper portion or main body 2104, which includes a hollow housing and the electronic and mechanical components of the delivery device 2100. Such components include a printed circuit board 2200 and associated components coupled thereto, a pair of batteries 2106, a hollow conduit, tube, or pipe 2108, a piezo-electric device 2110, which can include or be physically coupled to a mesh screen having a mesh size of 3 microns, of 4 microns, of 6 microns, of 20 microns, or of between 3 and 20 microns, as well as an internal cover 2112, and an external cover 2114, which can be transparent or translucent. The housing of the main body 2104 can be opaque or translucent, and can have a specific color such as red, orange, yellow, green, blue, purple, brown, black, or white. The internal cover 2112 can have an appearance matching that of the housing of the main body 2104. In particular, the internal cover 2112 can be opaque if the housing of the main body 2104 is opaque or translucent if the housing of the main body 2104 is translucent, and can have a specific color matching that of the housing of the main body 2104, such as red, orange, yellow, green, blue, purple, brown, black, or white.

The conduit 2108 includes a relatively wide top end portion, a relatively narrow middle portion and a relatively wide bottom end portion sized to extend around the conduit 2116 of the base 2102. An inner surface of the bottom end portion of the conduit 2108 includes threads complementary to the threads of the conduit 2116 so that the conduits 2108 and 2116 can be threadedly engaged and thereby coupled to one another. When the conduits 2108 and 2116 are coupled to one another, liquid scent media can be poured out of the base 2102 through the conduit 2116 and into the conduit 2108. The relatively wide top end portion of the conduit 2108 is sized and configured to house the piezo-electric device 2110 at the top end of the conduit 2108, so that the liquid scent media can flow through the conduit 2108 from the bottom end portion thereof to the piezo-electric device housed at the top end portion thereof.

The conduit 2108 also includes a pair of flanges 2118 that are coupled to opposing outer side surfaces of the middle portion of the conduit 2108, and that extend laterally outward from the respective side surfaces as well as in a direction aligned with the overall length of the conduit 2108. The flanges 2118 each include a recess or cradle that is shaped and configured to cradle a portion of one of the batteries 2106, to partially restrain the batteries 2106 when the device 2100 is assembled. The internal cover 2112 includes a generally circular or disk-shaped main body portion and a hollow and truncated cone-shaped portion 2120 that extends upward from the main body portion. The main body portion of the internal cover 2112 includes a pair of openings or apertures 2122 that extend through the main body portion. Each of the apertures 2122 is sized and configured to cradle a portion of one of the batteries 2106, to partially restrain the batteries 2106 when the device 2100 is assembled. The external cover 2114 includes a generally circular or disk-shaped main body portion and an opening or aperture 2124 that extends through the main body portion. The aperture 2124 is sized and configured to fit snugly around a portion of the outer surface of the cone-shaped portion 2120 of the internal cover 2112 when the device 2100 is assembled.

FIGS. 21B, 21C, and 21D illustrate perspective, side, and cross-sectional side views, respectively, of the delivery device 2100. FIGS. 21E and 21F illustrate two different side views of the delivery device 2100 with the housing of the main body 2104 removed to reveal internal components of the main body 2104.

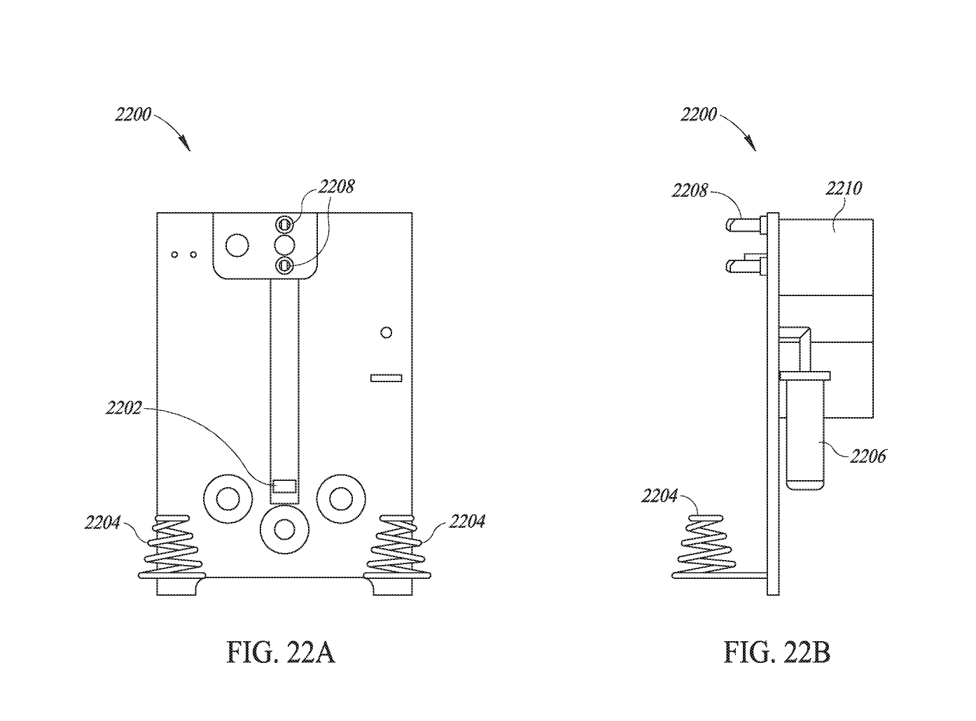

FIGS. 22A-22D illustrate the printed circuit board 2200 of the delivery device 2100 with associated components coupled thereto. FIG. 22A is a rear view of the printed circuit board 2200 and illustrates that the printed circuit board 2200 includes an LED 2202 physically and electrically coupled to the rear surface thereof, which can be operable to light up or turn on when the delivery device 2100 is generating a cloud of vaporized scent media or scent media in aerosol form, and to turn off when the delivery device 2100 is not generating a cloud of vaporized scent media or scent media in aerosol form. The LED can be useful to a user of the device 2100 because when the LED lights up, the user can be confident that power is being supplied to the printed circuit board 2200. FIG. 22A also illustrates that the rear surface of the printed circuit board 2200 is physically and electrically coupled to two metallic springs 2204, each of which is positioned and configured to act as a contact for, and to partially support or cradle, one of the batteries 2106. One of the springs 2204 can act as a positive contact, while the other of the springs 2204 can act as a negative contact, for the batteries 2106, such that the batteries 2016 will be installed within the device 2100 with their polarities reversed with respect to one another.

FIG. 22B is a side view of the printed circuit board 2200 and illustrates that the rear surface of the printed circuit board 2200 is also physically and electrically coupled to a plurality of gold pins 2208 to which a fluid sensor can be physically and electrically coupled. FIG. 22C is a front view of the printed circuit board 2200 and illustrates that the front surface of the printed circuit board 2200 can include an electrical connector 2212, which can be a JST connector, to allow an operator to physically and electrically couple other electronic devices, such as the piezo-electric device 2110, to the printed circuit board 2200 and to allow the printed circuit board and other associated components coupled thereto to communicate with (e.g., transmit signals to or receive signals from) such other electronic devices including the piezo-electric device 2110. FIG. 22C also illustrates that the front surface of the printed circuit board 2200 is physically and electrically coupled to a tilt sensor 2206, which can include an accelerometer or a ball tilt switch in which a ball moves and connects pins to complete an electrical circuit when the device 2100 is tilted, and to a plurality of capacitors 2210 for storing electrical energy. FIG. 22D is a perspective view of the printed circuit board 2200 and illustrates a perspective view of the printed circuit board 2200 with the associated components coupled thereto. FIGS. 22E-22G illustrate the printed circuit board 2200 without the associated components coupled thereto.

As illustrated in FIGS. 21A-21F, the rear of the printed circuit board 2200, illustrated directly in FIG. 22A, faces toward the conduit 2108 and the center of the delivery device 2100, while the front of the printed circuit board 2200, illustrated directly in FIG. 22C, faces away from the conduit 2108 and the center of the delivery device 2100. In some implementations, the printed circuit board 2200 receives power from a source at between 2.0 and 3.4 Volts DC, and provides power to a load at 140 KHz and at 65 Volts peak-to-peak. FIG. 22H illustrates a front view of an alternative shape and configuration for the printed circuit board 2200. FIGS. 22A-22H illustrate some examples of possible dimensions of the printed circuit board 22, with the numbers used in millimeters. It will be understood that the specific dimensions provided in these Figures are merely examples of possible suitable dimensions.

To operate the delivery device 2100, a user can fill the base 2102 with scent media in a liquid form and assemble the device 2100 except for the batteries 2106 and the external cover 2114, such as by screwing or threading the base 2102 onto the main body 2104. The user can then insert the batteries 2106 into the device 2100 through the apertures 2122 in the internal cover 2112, such that the batteries are partially cradled by the recesses of the flanges 2118, and such that bottom terminals of the batteries 2106 are in electrical contact with the springs 2204. The user can then couple the external cover 2114 to the rest of the device 2100, such as by threading or press-fitting the external cover into a top end of the main body 2104. An underside of the external cover 2114 can include a strip of electrically-conductive material, such as metal, which can engage the top terminals of the batteries 2106 and electrically couple the upper terminal of one of the batteries 2106 to the upper terminal of the other one of the batteries 2106.

The user can then lift and tilt the device 2100, such that the fluid flows, under the force of gravity, from the base 2102, through the conduit 2108, to the piezo-electric device 2110. Once the user tilts the device 2100, the tilt sensor 2206 can generate and transmit a signal indicating that the device 2100 has been tilted. Further, once the fluid flows to the piezo-electric device 2110, the fluid may come into contact with a fluid sensor coupled to the pins 2208 and generate and transmit a signal indicating that the fluid has reached the fluid sensor. Further still, the device 2100 can include a pressure-sensitive switch on a bottom surface thereof which, when the device 2100 is picked up off of a flat surface, can generate and transmit a signal that the device 2100 has been picked up. In some embodiments, the device 2100 includes no manually-operated switches or buttons, and receives no input from the user, other than one, two, or three of the signals described above.

Upon receipt of any one, any two, or all three of such signals, the device 2100 can activate the piezo-electric device 2110 to begin generating a cloud of vaporized scent media or scent media in aerosol form from the scent media in liquid form. Because the device 2100 is tilted sideways or upside-down, the cloud of vaporized scent media or scent media in aerosol form can flow out of the device 2100 through the hollow cone-shaped portion 2120, and can be consumed directly by the user or can be poured into another container or vessel for subsequent consumption. In some implementations, the device 2100 includes an internal timer and automatically turns off or de-activates the piezo-electric device 2110 to stop generating the cloud of vaporized scent media or scent media in aerosol form after a time period of about 5, about 10, about 15, or about 20 seconds. In other implementations, the device 2100 continues to operate and generate the vaporized scent media or scent media in aerosol form until the device 2100 is once again oriented upright or placed back on a flat horizontal surface.

When the fluid within the base 2102 runs out, the user can unscrew or unthread of the base 2102 from the main body 2104 of the device 2100, refill the base 2102 with more of a desired scent media in a fluid form, screw or thread the base 2102 back on to the main body 2104, and then resume using the device 100. When the batteries 2106 die, no longer power the device 2100, and need to be replaced, the user can remove the external cover 2114 from the rest of the device 2100, such as by unscrewing, unthreading, or turning the external cover 2114 with respect to the rest of the device 2100. The old batteries 2106 within the device 100 can then be removed and new batteries 2106 can be installed in their place. The user can then re-install the external cover 2114 onto the rest of the device 2100 and resume using the device 2100.

In some implementations, the external cover 2114, or a surface of the rest of the device 2100 that engages with the external cover 2114, includes a detent, and the detent is engaged as the external cover 2114 is turned with respect to the rest of the device 2100 just before the external cover 2114 is released from the rest of the device 2100. Engagement of the detent can serve as a signal to the user that the external cover 2114 is about to be released from the rest of the device 2100. Once the user releases and removes the external cover 2114 from the rest of the device 2100, the batteries are disconnected and the device is unable to operate. Thus, the external cover 2114 can act as a switch, where removing the external cover 2114 from the rest of the device 2100 switches the device 2100 off and engagement of the external cover 2114 with the rest of the device 2100 switches the device 2100 on.

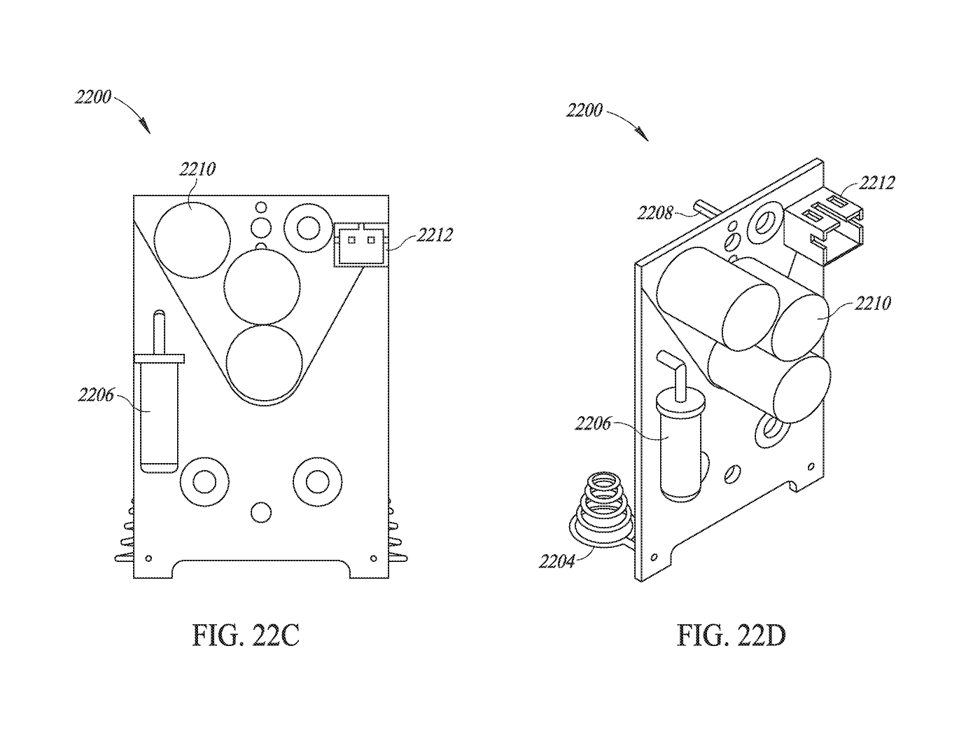

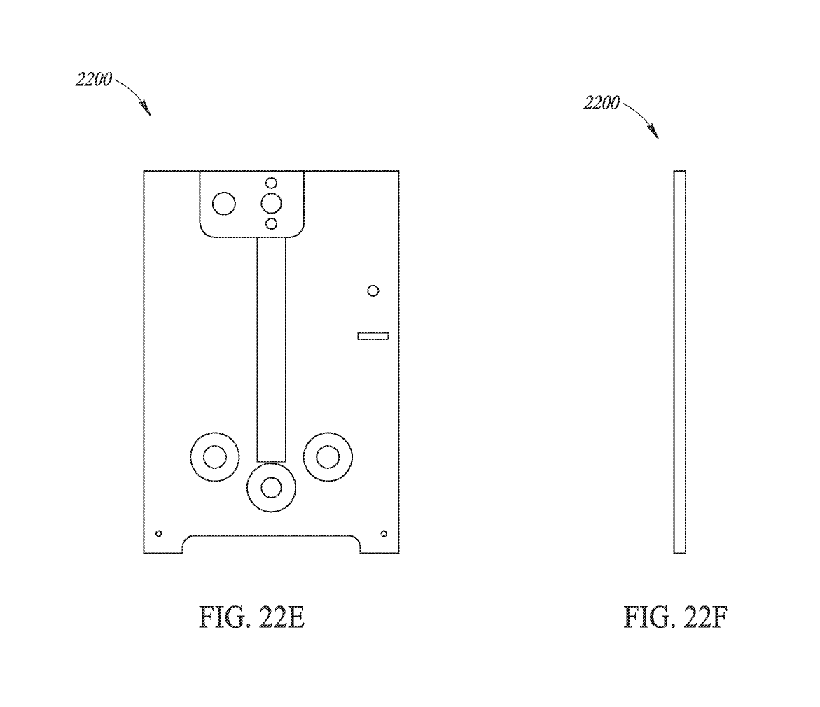

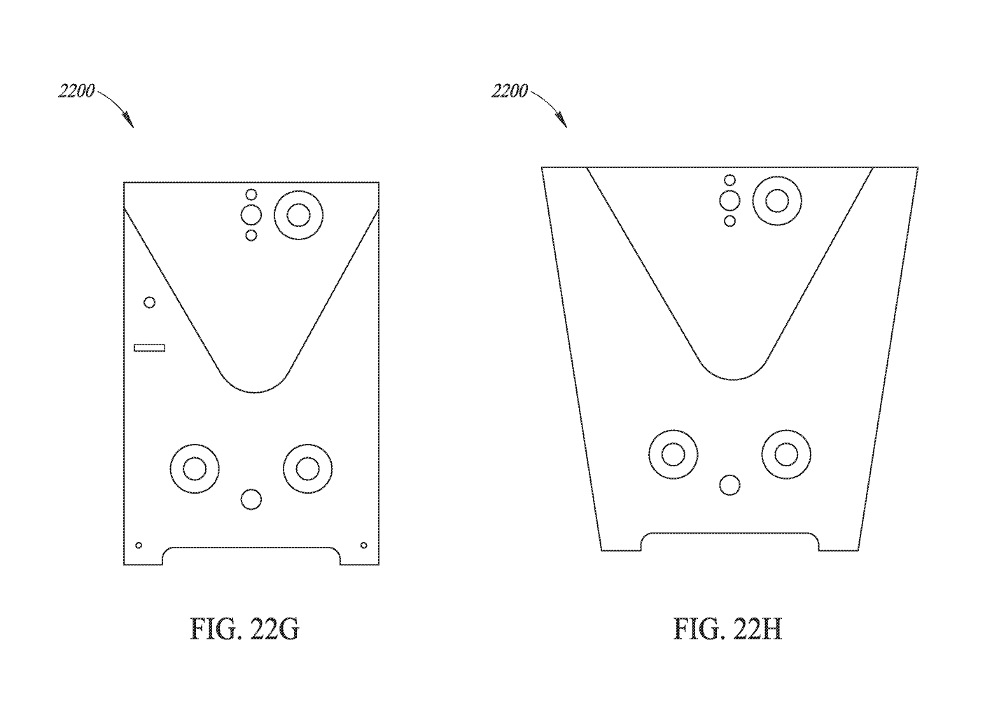

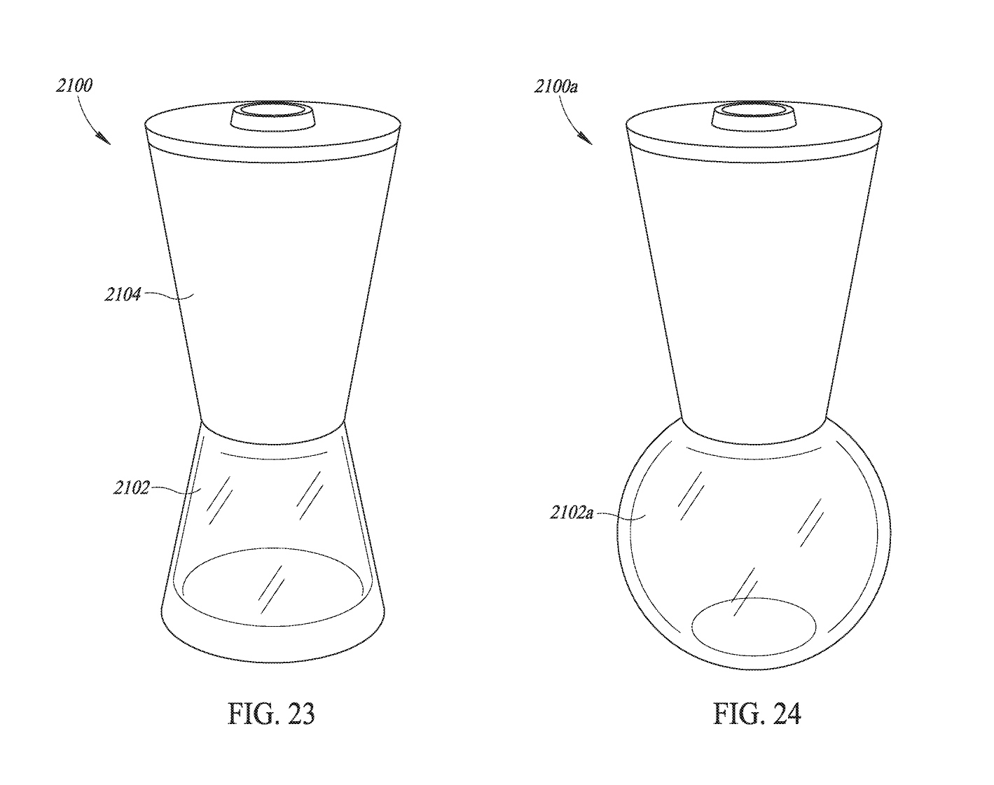

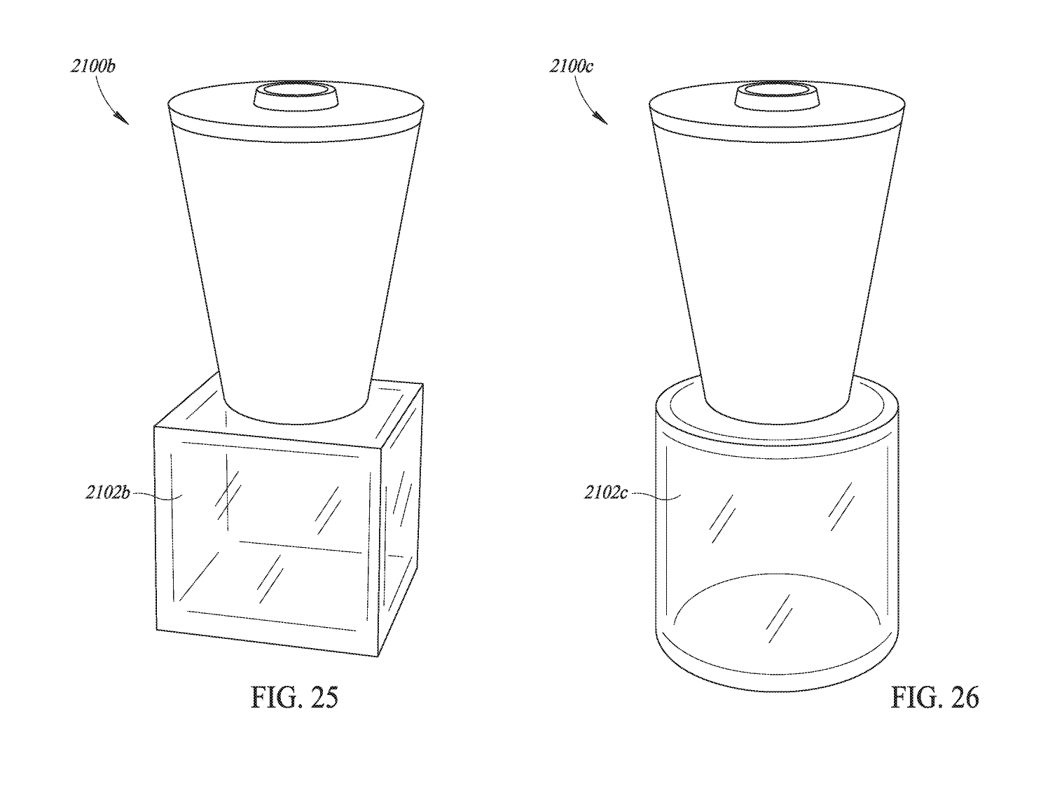

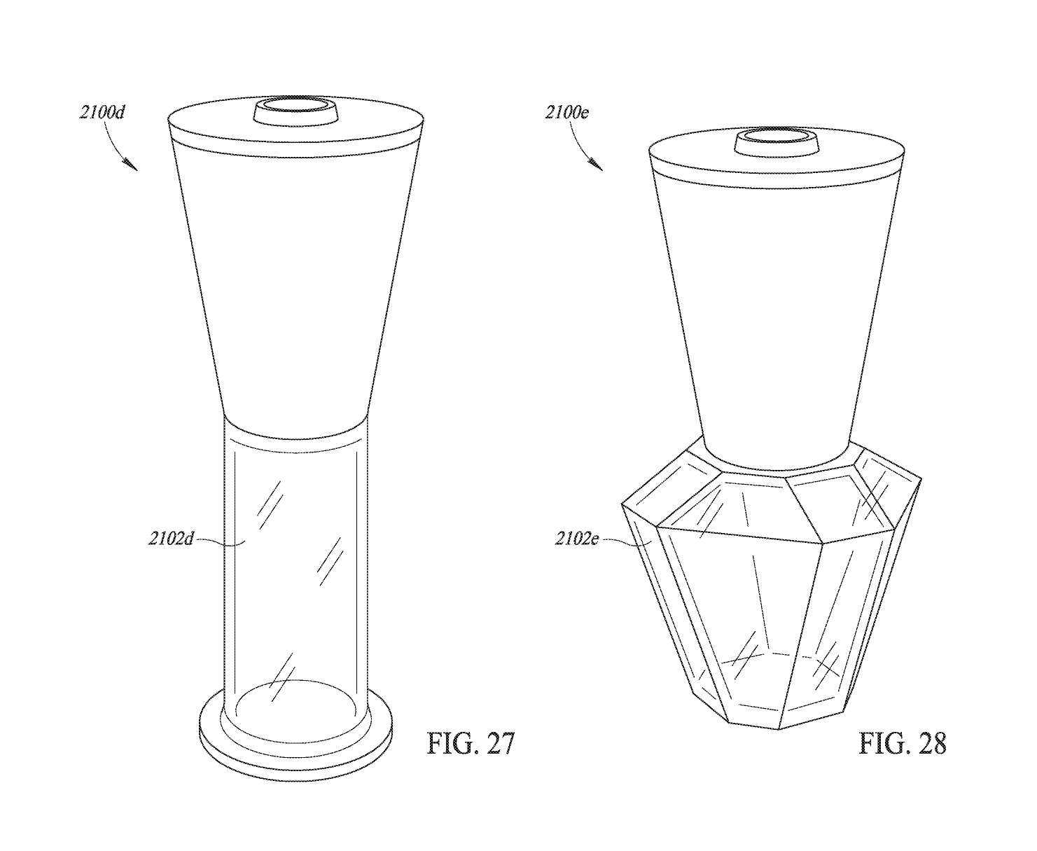

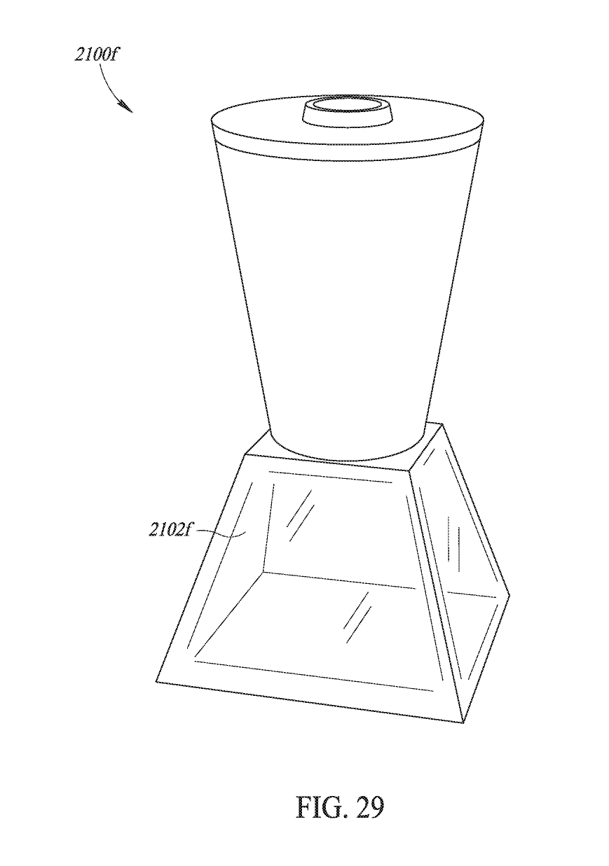

FIG. 23 illustrates a three-dimensional rendering of the device 2100, showing its overall shape. In particular, the base 2102 of the device 2100 has a geometric shape including a truncated cone, and the main body 2104 of the device 2100 has a geometric shape including an inverted truncated cone, such that the device 2100 has an overall geometric shape resembling an hourglass, with a bottom portion that is smaller than its top portion. FIG. 24 illustrates one alternative implementation of the device 2100a, in which the base 2102a has a geometric shape including a sphere with a truncated bottom end and a truncated top end. FIG. 25 illustrates another alternative implementation of the device 2100b, in which the base 2102b has a geometric shape including a cube. FIG. 26 illustrates another alternative implementation of the device 2100c, in which the base 2102c has a geometric shape including a relatively short and wide cylinder. FIG. 27 illustrates another alternative implementation of the device 2100d, in which the base 2102d has a geometric shape including a relatively tall and narrow cylinder. FIG. 28 illustrates another alternative implementation of the device 2100e, in which the base 2102e has a geometric shape resembling a truncated diamond, such as resembling a truncated asscher-cut diamond. FIG. 29 illustrates another alternative implementation of the device 2100f, in which the base 2102f has a geometric shape including a truncated four-sided pyramid.

In some implementations, the scent media used in any one of the delivery devices described herein can include functional additives or active agents, in some cases for therapeutic applications, such as vitamins, minerals, supplements, other nutrients to provide nutrition, or drugs, medications, or pharmaceutical agents. As specific examples, such compounds can be used to assist a user in recovering from addictions such as opiate or food addictions, or to assist a user in controlling their metabolism. In some cases, such compounds can be provided in a liquid, and either a concentrated or a purified form, and can be vaporized or aerosolized by one of the delivery devices described herein and poured over a food or a beverage that either does not contain such compounds or that contains such compounds in a much lower concentration than the vapor or aerosol.

As one specific example, a commercial syrup, such as a commercial cinnamon syrup, such as is commercially available under the brand name Torani, can be diluted with water in a ratio of between one third and one half syrup to between one half and two thirds water, and then mixed with a water-soluble vitamin such as vitamin B12 such that the vitamin makes up about five percent of the mass of the mixture. The mixture can then be poured into a vial within one of the delivery devices described herein, and the delivery device can be operated to vaporize or aerosolize the mixture and create a cloud of the vapor or aerosol over a food or a beverage such as a glass half-full of water. In such an implementation, the mass of the cloud over the water is around 1 milligram and the cloud contains about 50 micrograms of the vitamin. In this way, a user, such as a patient, can consume a daily dose of vitamin B12 by consuming ten sips of water if with each sip, the user consumes the cloud of vapor or aerosol over the water and then pours a new cloud of vapor or aerosol for consumption with the next sip of water.

As another example, the same process can be performed but with a peppermint syrup and with vitamin D mixed into the syrup and water such that the vitamin makes up about one percent of the mass of the mixture. In such an implementation, the cloud of vapor or aerosol over the water contains about 10 micrograms of the vitamin. In this way, a user, such as a patient, can consume a daily dose of vitamin D by consuming two sips of water if with each sip, the user consumes the cloud of vapor or aerosol over the water and then pours a new cloud of vapor or aerosol for consumption with the next sip of water.

As another example, the same process can be performed but with a salty caramel syrup and with folic acid mixed into the syrup and water such that the folic acid makes up about five percent of the mass of the mixture. In such an implementation, the cloud of vapor or aerosol over the water contains about 50 micrograms of folic acid. In this way, a user, such as a patient, can consume a daily dose of folic acid by consuming eight sips of water if with each sip, the user consumes the cloud of vapor aerosol over the water and then pours a new cloud of vapor or aerosol for consumption with the next sip of water.

This process can be adjusted for use with a wide variety of vitamins, minerals, other nutrients, or medications, including to deliver a 700-900 μg dose of Vitamin A (RAE), a 300 μg dose of Biotin, a 80 μg dose of Vitamin K, a 120 μg dose of Chromium, a 150 μg dose of Iodine, a 75 μg dose of Molybdenum, or a 70 μg dose of Selenium to a user such as a patient. This process can be used to create clouds of vaporized or of droplets of aerosolized compounds where the total mass of the droplets in the cloud is, or is about, or is less than or equal to, 1 milligram.

U.S. provisional patent application Nos. 62/652,069, filed Apr. 3, 2018, 62/628,395, filed Feb. 9, 2018, and 62/556,974, filed Sep. 11, 2017 and 62/687,970, filed Jun. 21, 2018, are hereby incorporated by reference, in their entireties. The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-2. (canceled)

3. The delivery system of claim 43 wherein the vessel has at least one opening through which the aerosol is accessible by a user, the at least one opening is sized and dimensioned to accommodate a portion of a nose including two nostrils.

4-15. (canceled)

16. The scent delivery system of claim 43 further comprising the active substance, wherein the readily-soluble droplets are water droplets and the active substance is dissolved in the water droplets.

17-18. (canceled)

19. The scent delivery system of claim 43 further comprising the active substance, wherein the active substance is in the form of readily-soluble droplets of the active substance.

20. The scent delivery system of claim 43 further comprising the active substance, wherein the active substance is encapsulated inside the droplets.

21. The scent delivery system of claim 43 further comprising the active substance, wherein the active substance is formulated as micelles.

22. The scent delivery system of claim 43 further comprising the active substance, wherein the active substance is formulated as micro-emulsions.

23. The scent delivery system of claim 43 further comprising the active substance, wherein the active substance is formulated as emulsions.

24. The scent delivery system of claim 43 further comprising the active substance, wherein the active substance is formulated as liposomes.

25. The scent delivery system of claim 43 further comprising the active substance, wherein the active substance is formulated as nanoparticles.

26. The scent delivery system of claim 43 further comprising the active substance, wherein the active substance is formulated as colloids.

27-42. (canceled)

43. A delivery system, the delivery system comprising:
a vessel having at least one side wall which at least partially delimits an interior of the vessel from an exterior thereof;
a media reservoir that at least in use holds active substance media;
an actuator controllably operable on the active substance media to cause formation of an aerosol comprising readily-soluble droplets have a median size range of approximately 2 microns to approximately 10 microns and comprising the one or more active substances; and
a control subsystem communicatively coupled to control the actuator.

44. (canceled)

45. The delivery system of claim 43 wherein the vessel has a top and the actuator or a portion thereof is positioned relatively below the top of the vessel.

46. The delivery system of claim 45 wherein the actuator or a portion thereof is positioned relatively below the top of the vessel, and the vessel has an opening at least proximate the top of the vessel and the actuator or a portion thereof is oriented to dispense the aerosol toward the at least one side wall of the vessel perpendicular to at least a portion of the at least one side wall of the vessel and/or at a downward angle with respect to the top of the vessel.

47. The delivery system of claim 46 wherein the vessel has an opening at least proximate the top of the vessel, and further comprising:
a filter positioned across an opening of the fluidly communicative scent path, the filter includes a mesh screen having mesh openings of no greater than 500 micrometers.

48-51. (canceled)

52. The delivery system of claim 45 wherein the vessel has an opening at least proximate the top of the vessel, and further comprising:
a cover that is detachable coupleable to the vessel to selectively deny access into the interior of the vessel from the exterior thereof when the cover is positioned over the top of the vessel and to provide access into the interior of the vessel from the exterior thereof when the cover is removed from the top of the vessel.

53. The delivery system of claim 52, further comprising:
a hollow conduit that provides a fluidly communicative path between the interior of the vessel and the exterior thereof when the cover is positioned over the top of the vessel.

54. (canceled)

55. The delivery system of claim 43, further comprising:
a base on which the vessel is support, the vessel removably supported by the base, and at least a portion of the control subsystem is housed by the base.

56-60. (canceled)

61. The delivery system of claim 55, further comprising:
at least one of a switch or a sensor communicatively coupled to the control subsystem and responsive to a presence or an absence of the vessel with respect to the base and operable to produce a signal that causes the control subsystem to operate the actuator according to the presence or an absence of the vessel with respect to the base.

62. The delivery system of claim 43, further comprising:
at least one of a switch or a sensor communicatively coupled to the control subsystem and responsive to a position or orientation of the vessel and operable to produce a signal that causes the control subsystem to operate the actuator according to the orientation of the vessel.

63. The delivery system of claim 43, further comprising:
at least one receiver that receives signals from an external source, the receiver communicatively coupled to the control subsystem and operable to produce a signal that causes the control subsystem to operate the actuator according to the signals received from the external source.

64-66. (canceled)

67. The delivery system of claim 43, further comprising:
at least one of a switch communicatively coupled to the control subsystem, wherein the at least one switch is a momentary contact switch operable by contact with a portion of a vessel into which the conduit is located or by contact with a portion of a hand of a user of the delivery system.

68. The delivery system of claim 43, wherein the media reservoir is a removable scent media cartridge that contains the active substance media.

69-93. (canceled)

94. The delivery system of claim 43, further comprising the active substance, wherein:
the active substance includes a nutritional compound or a medication; and
wherein a flavor of the flavored active substance media corresponds to a food or a beverage item; and
wherein the flavored active substance media can be consumed retro-nasally.

95-97. (canceled)

98. A method of administering an aerosolized composition to a person in need thereof, the method comprising:
aerosolizing a flavored active substance media including a nutritional compound or a medication to create the aerosolized composition;
wherein the aerosolized composition comprises droplets having a median size between 2 microns and 10 microns;
wherein a total mass of the droplets of the aerosolized composition is less than or equal to 1.0 milligrams;
wherein a flavor of the flavored active substance media corresponds to a food or a beverage item; and
wherein the flavored active substance media can be consumed retro-nasally.
